(19) United States
(12) Patent Application Publication
(10) Pub. No.: US 2026/0137603 A1
(43) Pub. Date: May 21, 2026

(54) MULTILAYER MAKEUP PROTECTIVE COSMETIC

(71) Applicant: KOSE Corporation, Tokyo (JP)

(72) Inventor: Kaoru KATO, Tokyo (JP)

(73) Assignee: KOSE Corporation, Tokyo (JP)

(21) Appl. No.: 19/131,648

(22) PCT Filed: Dec. 25, 2023
(86) PCT No.: PCT/JP2023/046373

§ 371 Date: May 21, 2025

(30) Foreign Application Priority Data
Dec. 28, 2022 (JP) 2022-211611

(51) Int. Cl. A61K 8/03; A61Q 1/02

(52) U.S. Cl. CPC A61K 8/03; A61Q 1/02; A61K 2800/594; A61K 2800/95

(57) ABSTRACT

Provided is a multilayer makeup protective cosmetic that has good film formation speed when used, has a good feel of use without stickiness and is capable of preventing clogging of a spray opening or spray nozzle occurring over time. The present invention can provide a multilayer makeup protective cosmetic containing ingredients (A) to (D). (A) A lipophilic resin. (B) Ethanol. (C) A metal salt. (D) A low-viscosity non-volatile oil agent having a kinematic viscosity of less than 300 mm²/s at 25° C.

### TECHNICAL FIELD

The present technique relates to a multilayer makeup protective cosmetic and the like.

### BACKGROUND ART

There is a consumer's desire to prevent a makeup smudging caused by sweat or rubbing against clothes after makeup.

For example, Patent Literature 1 discloses that a method for providing an overcoat agent for preventing smudging, according to which secondary makeup is applied mainly to, among eye makeup cosmetics, cosmetics for eyes and eyebrows mainly used for eye edges, eyelashes, and eyebrows, such as eyeliners, mascaras, and eyebrow pencils, thereby achieving significant improvement in makeup durability and secondary adhesion (claim 1).

Patent Literature 2 proposes a water-based or oil-in-water type mist cosmetic which contains (A) a specific water-soluble thickening agent and which is filled in a spray pump container having an aperture of φ 0.25 to φ 0.55 mm (claim 1, claim 3, paragraph [0024], [Table 1]).

For example, Patent Literature 3 discloses to provide a skin cosmetic especially in the form of a lotion or a cream which is used over the foundation with makeup to moist the skin, which is also a skin cosmetic that has excellent long-term stability, does not let the foundation go patchy even when applied over the face with makeup, and is excellent in terms of a sense of moistness over time (paragraphs [0001], [0002]).

For example, Patent Literature 4 proposes, in its claim 1, a multi-layer makeup protection cosmetic for use by spraying over makeup, comprising the following components (A) to (C): (A) an oil-soluble film-forming agent, (B) 40 to 95 mass % of water and (C) a volatile oil agent.

### CITATION LIST

### Patent Literature

Patent Literature 1: JP-A2007-320905
Patent Literature 2: JP-A2014-237615
Patent Literature 3: JP-A2002-138030
Patent Literature 4: WO2020/175398

### SUMMARY OF INVENTION

### Technical Problem

A main object of the present invention is to provide a multi-layer makeup protective cosmetic that has good film formation speed when used, has a good feel of use without stickiness and is capable of preventing clogging of a spray opening or spray nozzle occurring over time.

### Solution to Problem

As a result of extensive researches conducted by the present inventors, it was found that a multi-layer makeup protective cosmetic that has good film formation speed when used, has a good feel of use without stickiness and is capable of preventing clogging of a spray opening or spray nozzle occurring over time can be provided by forming a composition containing ingredients (A) a lipophilic resin, (B) ethanol, (C) a metal salt and (D) a low-viscosity non-volatile oil agent, thus accomplishing the present invention. The present invention is as follows.

The present invention can provide a multilayer makeup protective cosmetic containing ingredients (A) a lipophilic resin, (B) ethanol, (C) a metal salt and (D) a low-viscosity non-volatile oil agent having a kinematic viscosity of less than 300 mm²/s at 25° C.

### Advantageous Effects of Invention

According to the present invention, a multi-layer makeup protective cosmetic that has good film formation speed when used, has a good feel of use without stickiness and is capable of preventing clogging of a spray opening or spray nozzle occurring over time can be provided.

### DESCRIPTION OF EMBODIMENTS

Hereinafter, preferred embodiments for conducting the present technique will be described. The embodiments described hereinafter show examples of typical embodiments of the present technique, and do not narrow the interpretation of the scope of the present technique. Unless otherwise noted, a percentage is expressed here based on the mass (% by mass). The upper limit (not more than xx) and the lower limit (not less than yy) of each numerical range (yy to xx) can be arbitrarily combined as required.

### 1. Present Technique

The present inventors conducted extensive researches for a multilayer protective composition hoped to have a makeup protection effect which is a multilayer protective composition containing an oil-based resin. Since the present technique is a multilayer composition, the condition of the appearance of the composition is different from those of the cosmetics of Patent Literatures 1 to 3, and it is a matter of course that the concept or approach in combining the components is different substantially.

With regard to a multilayer makeup protective cosmetic employing an oil-soluble film-forming silicone-based resin, the present inventors discovered that a multilayer composition containing an oil-soluble film-forming silicone-based resin together with 40 to 95% of water and a volatile oil agent exhibits good mist fineness and makeup protection effect but also exhibits a stickiness after application and that continuous use of the spray container allows a lipophilic resin to be precipitated near the discharge port, resulting in a clogging of the spray opening or the spray nozzle. The present inventors considered that when a volatile oil agent is used in a composition the film forming speed when used is good while clogging of the spray opening or the spray nozzle is allowed to occur over time. The present inventors also discovered, as shown in Reference Example 1 in [Examples] shown below, that a composition containing a lipophilic resin, ethanol, water and a volatile oil agent, which is formulated additionally with ethanol, still allowed clogging of the spray opening or the spray nozzle to occur over time.

Accordingly, a main object of the present technique is to provide a multi-layer makeup protective cosmetic that has good film formation speed when used, has a good feel of use without stickiness and is capable of preventing clogging of a spray opening or spray nozzle occurring over time.

Moreover, as a result of extensive researches conducted by the present inventors, it became possible to provide a multilayer makeup protective cosmetic which has a good feel of use without stickiness and is capable of preventing clogging of a spray opening or spray nozzle occurring over time by forming a composition containing a lipophilic resin, ethanol, a metal salt and a low-viscosity non-volatile oil agent (Examples 1 to 28). Furthermore, the present technique also has an advantage in that it achieves a good film forming speed of the lipophilic resin even when using no volatile oil agent (Examples 1 to 4 etc.). Moreover, the present technique also has an advantage in that it can provide a multilayer makeup protective cosmetic that has good film formation speed when used, has an excellent feel of use without stickiness and is capable of preventing clogging of a spray opening or spray nozzle occurring over time, even when additionally containing a non-volatile oil agent having a middle-to-high viscosity which is different from a low-viscosity non-volatile oil agent (Examples 25 and 26). Moreover, the present technique also has an advantage in that it can provide a multilayer makeup protective cosmetic that has an excellent feel of use without stickiness and is capable of preventing clogging of a spray opening or spray nozzle occurring over time and also has good film formation speed when used, even when additionally containing a volatile oil agent (Examples 27 and 28). The composition of the present technique may be used as a cosmetic or a dermal preparation, and may employ the ingredients, the constitutions, the contents and the like detailed below as appropriate.

The present technique can also provide a multilayer cosmetic having a makeup protection effect, and the cosmetic may be a dermal preparation or a composition. The present technique also has an advantage in that it can be used not only over a makeup as conducted usually by a user but also over the skin before applying the makeup or even in the course of applying the makeup, thereby preventing or inhibiting usual smudging of the makeup thus exhibiting the makeup protection effect. As a result, it exhibits a preventive effect against secondary adhesion onto clothes or facemasks. Furthermore, also in the case of applying after mixing with a cosmetic such as foundation, blush, eye shadow and the like, or applying the cosmetic which was poured preliminarily onto a cosmetic applicator such as a brush or a sponge, usual makeup smudging can be prevented or inhibited, thereby exhibiting makeup the protection effect. Accordingly, the present technique also serves to respond to consumers' demand for improving the lasting of their individual unique makeup applied onto skin or face using their own usual cosmetics. Furthermore, the present technique can be applied or employed by a user as appropriate depending on makeup condition, approach, retouching (makeup retouching), procedure, approach or technique over a desired makeup, or during or after conducting a desired makeup. The present technique can be used at the time of using a cosmetic also by spraying directly to clothes or facemask and the like which is brought into contact with a skin to prevent secondary adhesion of the makeup onto the clothes or facemask and the like thereby achieving the makeup protection effect. While various modes of use can be employed, it is preferable from the viewpoint of the makeup protection effect to use onto the makeup, onto skin before applying the makeup or use during makeup.

Also the present inventors found that by using a lipophilic resin a film having an excellent barrier effect against pollen and air pollutant particles such as PM2.5 and yellow sand can readily be formed (Patent Literature 4: WO2020/175398). Since smudging is prevented over a prolonged time period by using a multilayer composition containing at least a lipophilic resin onto the makeup as described below in [Examples], the present technique can be used not only for the purpose of makeup protection, but also as a composition for the purpose of skin protection, such as for blocking air pollution or as a measure against pollen or PM2.5, airborne particles and the like by using over skin before or after applying makeup. The present technique also has an advantage in that it becomes difficult for various particles to adhere onto the surface of the skin or the surface of the makeup after using a skin protective composition relating to the present technique.

The present technique is detailed below.

### 2. Multilayer Makeup Protective Cosmetic According to Present Embodiment

The present embodiment preferably provides a multi-layer makeup protective cosmetic containing ingredient (A) lipophilic resin, ingredient (B) ethanol, ingredient (C) metal salt and ingredient (D) a low-viscosity non-volatile oil agent, and the low viscosity of the ingredient (D) is preferably a kinematic viscosity of less than 300 mm²/s at 25° C. As a result, a good feel of use without stickiness and a preventive effect on clogging of a spray opening or spray nozzle over time can be exerted. In addition, it is further advantageous in that the film forming speed when used is good.

Also, the makeup protective cosmetic can be used not for makeup protection but as a skin protective composition or a skin protective cosmetic. The makeup protective cosmetic may be used also as a protective measure against, or for preventing adhesion of airborne particles such as pollen or PM2.5.

It is preferable to use the present embodiment by spraying onto the face. The present embodiment is advantageous in that it allows a uniform film to be formed readily over the face or the makeup since it can spray a fine mist, and accordingly it is preferable from the viewpoint of makeup protection.

In the present embodiment, the ingredient (A) lipophilic resin is preferably a silicone-based resin. Also in the present embodiment, the ingredient (C) is preferably a salt of an alkaline metal or a salt of an alkaline earth metal, a metal salt of an inorganic acid. Also in the present embodiment, the ingredient (D) is preferably one or more kinds selected from the group consisting of a silicone oil, an ester oil and a hydrocarbon oil. Also in the present embodiment, it is preferable that water and/or volatile oil agent are further contained. Also in the present embodiment, the mass content ratio (B)/(A) of the ingredient (B) to the ingredient (A) is preferably 1 to 200. Also in the present embodiment, the mass content ratio (C)/(A) of the ingredient (C) to the ingredient (A) is preferably 0.02 to 20. In the present embodiment, the mass content ratio (A)/(D) of the ingredient (A) to the ingredient (D) is preferably 0.01 to 6. Also in the present embodiment, it is preferable that volatile oil agent is further contained. Also in the present embodiment, a non-volatile oil agent having a middle-to-high viscosity may further be contained.

A multilayer makeup protective cosmetic according to the present embodiment is detailed below. Each ingredient employed in the present embodiment is not limited particularly as long as it can be employed ordinarily in a cosmetic or a dermal preparation.

### <2-1. Lipophilic Resin>

While the lipophilic resin (hereinafter also referred to as “ingredient (A) lipophilic resin” or “ingredient (A)”) employed in the present embodiment is not limited particularly as long as it can be used ordinarily in a cosmetic or a dermal preparation, it is preferably a lipophilic resin which has an ability of being dissolved in an oil agent or swollen by an oil agent to form a uniform dispersion and which can be used in an ordinary cosmetic or dermal preparation. Such a lipophilic resin is preferable from the viewpoint of makeup protection. The lipophilic resin is more preferably an oil-soluble film-forming lipophilic resin, and this oil-soluble film-forming lipophilic resin is soluble in an oil agent such as a volatile oil and the like and can form a film, and a resin which can be used in an ordinary cosmetic or dermal preparation is further preferred.

As used herein, the “resin” in “a lipophilic resin” means to include a tacky substance derived from a tree bark and a polymer compound, and may for example be a naturally-occurring resin, a semi-synthetic resin, a synthetic resin and the like regardless of the origins, which may be used ordinarily in a cosmetic or a dermal preparation. As used herein, the “lipophilic” of “a lipophilic resin” denotes an ability of being dissolved in an oil agent or being swollen by an oil agent to form a uniform dispersion.

The ingredient (A) lipophilic resin is not limited particularly, and may for example be, a silicone-based resin such as polymethyl silsesquioxane, polypropyl silsesquioxane, trimethylsiloxy silicate, trifluoroalkyldimethyl trimethylsiloxy silicate, (trimethylsiloxy silicate/dimethiconol) cross-polymer, (dimethicone/vinyl trimethylsiloxy silicate) cross-polymer, (dimethicone/vinyl dimethicone) cross-polymer, (vinyldimethyl/trimethylsiloxy silicate/dimethicone) cross-polymer, (vinyldimethyl/trimethylsiloxy silicate stearyl dimethicone) cross-polymer and the like; a (meth)acrylate-based resin such as (styrene/acrylates) copolymer, (cyclohexyl methacrylate/ethylhexyl methacrylate) copolymer and the like; a (meth)acryl-silicone graft polymer such as (acrylates/dimethicone) copolymer, (acrylates/polytrimethylsiloxy ethacrylate) copolymer and the like; a dextrin fatty acid ester such as dextrin isostearate and the like; an α-olefin/vinylpyrrolidone copolymer such as (VP/Eicosene) copolymer and the like; a silicone-derived polysaccharide compound such as tri(trimethylsiloxy)silylpropyl carbamate pullulan and the like; a pullulan fatty acid ester such as pullulan myristate, pullulan palmitate and the like; a rosin acid-based resin such as rosin-modified phenol resin, rosin ester and the like; a candelilla resin; a vinyl acetate-based resin; a saturated or unsaturated hydrocarbon resin of butene-based resin etc. such as polybutene resin, polyisoprene resin, hydrogenated resin thereof and the like; and polyurethan resin and the like. These ingredients (A) allow the film formation speed, good feel of use without stickiness, prevention of the spray opening or spray nozzle from clogging over time to be good readily by combining the ingredients described below. One or more kinds can be selected from these ingredients (A), and combination of multiple kinds is preferable. As used herein, “(meth)acrylic acid” means “acrylic acid” and/or “methacrylic acid”, and “(meth)acryl” means acryl and/or methacryl.

From the viewpoint of makeup protection effect and the fineness of the mist when used, the ingredient (A) lipophilic resin is preferably one or more kinds selected from the group consisting of a silicone-based resin, (meth)acrylic acid-based resin, (meth)acryl-silicone graft polymer and dextrin fatty acid ester, more preferably one or more kinds selected from the group consisting of a silicone-based resin, (meth)acrylic acid-based resin and (meth)acryl-silicone graft polymer, among which a silicone-based resin is preferred. Although one selected from the examples of the lipophilic resins can solely be employed in the present embodiment, use of a combination of two kinds or more is more preferable because the makeup protection effect and the fineness of the mist when used become more excellent. Also for the purpose of allowing the feel of use without stickiness and the makeup protection effect to become further favorable, two or more kinds selected from the group consisting of silicone-based resins such as polymethyl silsesquioxane and the like listed above may be employed in combination, and the combination of polymethyl silsesquioxane and trimethylsiloxy silicate may for example be employed.

Among ingredients (A) lipophilic resins, one or more kinds selected from the group consisting of polymethyl silsesquioxane, trimethylsiloxy silicate, (acrylates/dimethicone) copolymer, trifluoroalkyldimethyl trimethylsiloxy silicate, (dimethicone/vinyl trimethylsiloxy silicate) cross-polymer, (trimethylsiloxy silicate/dimethiconol) cross-polymer, (dimethicone/vinyl dimethicone) cross-polymer, (stylene/acrylates) copolymer, (cyclohexyl methacrylate/ethylhexyl methacrylate) copolymer, dextrin isostearate is preferred, one or more kinds selected from the group consisting of (dimethicone/vinyl dimethicone) cross-polymer, polymethyl silsesquioxane, trimethylsiloxy silicate, (acrylates/dimethicone) copolymer, trifluoroalkyldimethyl trimethylsiloxy silicate, (dimethicone/vinyl trimethylsiloxy silicate) cross-polymer, (trimethylsiloxy silicate/dimethiconol) cross-polymer, (stylene/acrylates) copolymer, (cyclohexyl methacrylate/ethylhexyl methacrylate) copolymer, dextrin isostearate is more preferred, and one or more kinds selected from the group consisting of (dimethicone/vinyl dimethicone) cross-polymer, polymethyl silsesquioxane, trimethylsiloxy silicate, (acrylates/dimethicone) copolymer, trifluoroalkyldimethyl trimethylsiloxy silicate, (dimethicone/vinyl trimethylsiloxy silicate) cross-polymer, (trimethylsiloxy silicate/dimethiconol) cross-polymer is further preferred. As a result, the makeup protection effect and the fineness of the mist when used become more favorable, the film forming speed becomes more favorable, a good feel of use without stickiness is experienced, and the preventive effect on the clogging of the spray opening or spray nozzle over time can readily be achieved.

It is preferable that the ingredient (A) lipophilic resin contains at least trimethylsiloxy silicate, and in such a case the trimethylsiloxy silicate may be combined with other lipophilic resin or with another different silicone-based resin.

Among the ingredients (A) lipophilic resins, a film-forming lipophilic resin is preferable from the viewpoint of the makeup protection effect. The film formation means that, when a resin solution formed by dissolving a lipophilic resin in a volatile oil agent is applied uniformly over a glass plate in a certain thickness (for example 400 μm) and then the volatile oil agent is evaporated, a film is formed thereafter.

As a film-forming lipophilic resin employed in the present embodiment, a film-formable resin among the examples of the lipophilic resins and compound described above for the “ingredient (A) lipophilic resin” can appropriately be employed, and those employed preferably among such resins are a film-forming silicone-based resin, a film-forming (meth)acrylic acid-based resin, a film-forming (meth)acryl-silicone graft polymer and a film-forming dextrin fatty acid ester, with a film-forming silicone-based resin, a film-forming (meth)acrylic acid-based resin, a film-forming (meth)acryl-silicone graft polymer being more preferred. Among such resins, a film-forming silicone-based resin is preferable from the viewpoint of ability of allowing a film which is resistant to sweat and sebum and has no stickiness to be formed more favorably. One or more kinds selected therefrom can be used.

In the present embodiment, a commercially available product can be employed as the ingredient (A). For example, KF7312J (solids content: 50%, solvent: cyclopentasiloxane, manufactured by Shin-Etsu Chemical Co., Ltd.), KF-9021 (solids content: 50%, solvent: cyclopentasiloxane, manufactured by Shin-Etsu Chemical Co., Ltd.), BY11-018 (solids content: 30%, solvent: cyclopentasiloxane, manufactured by Dow Corning Toray Co., Ltd.), KP541 (solids content: 60%, solvent: isopropanol, manufactured by Shin-Etsu Chemical Co., Ltd.), SR-1000 (manufactured by Momentive Performance Materials Japan LLC.), KP545 (solids content: 30%, solvent: cyclopentasiloxane, manufactured by Shin-Etsu Chemical Co., Ltd.), KP575 (solids content: 30%, solvent: cyclopentasiloxane, manufactured by Shin-Etsu Chemical Co., Ltd.), BELSIL TMS 803 (manufactured by Wacker Asahikasei Silicone Co., Ltd.), BELSIL RG 90 (solid content 20%, solvent: isododecane, manufactured by Wacker Asahikasei Silicone Co., Ltd.), BELSIL RG 100 (solid content 20%, solvent: decamethyl cyclopentasiloxane, manufactured by Wacker Asahikasei Silicone Co., Ltd.), DOWSIL FC-5004DM (1.5 cSt) Silicone Resin Gum (solid content 40%, solvent: dimethicone, manufactured by Dow Corning Toray Co., Ltd.), SILFORM FLEXIBLE RESIN (manufactured by Momentive Performance Materials Japan LLC.), XS66-B8226 (solids content: 50%, solvent: cyclopentasiloxane, manufactured by Momentive Performance Materials Japan LLC.), OPPANOL B-100 (manufactured by BASF SE), UNIFILMA HVY (manufactured by Chiba Flour Milling Co., Ltd.), and the like can be mentioned. One or more kinds selected therefrom can be used.

While the content of the ingredient (A) lipophilic resin (preferably film-forming lipophilic resin) in the entire mass of the cosmetic is not limited particularly, the preferred lower limit thereof, from the viewpoint of the makeup protection effect, is preferably 0.01% or more, more preferably 0.05% or more, further preferably 0.1% or more, still more preferably 0.5% or more, more preferably 1% or more, and the preferred upper limit thereof, from the viewpoint of the feel of use without stickiness, is preferably 15% or less, more preferably 10% or less, further preferably 8% or less, still more preferably 5% or less. The preferred numerical range of the content of the ingredient (A) lipophilic resin in the entire mass of the cosmetic is preferably 0.01 to 10%, more preferably 0.05 to 8%. Within this range, the makeup protection effect can be experienced while allowing the feel of use without stickiness and the preventive effect on clogging of a spray opening or spray nozzle over time to be favorable and allowing the film forming speed when used to be adjusted favorably.

When using a combination of two or more different lipophilic resins, the content of the ingredient (A) described above may be the content of the entire ingredient (A). When using a combination of two different lipophilic resins, the mass content ratio ((a):(b)) thereof is preferably 10 to 1:1 to 10, more preferably 5 to 1:1 to 5, further preferably 2 to 1:1 to 2. It is preferable that the (a) described above is trimethylsiloxy silicate, and the (b) described above is other lipophilic resin or a silicone-based resin different therefrom.

While the silicone-based resin content is not limited particularly, the preferred lower limit thereof in the content of the ingredient (A) lipophilic resin is preferably 50% or more, more preferably 80% or more, still more preferably 90% or more, yet more preferably 95% or more, more preferably 99% or more, and more preferably substantially 100%.

While the silicone-based resin content in the entire composition mass is not limited particularly and the preferred lower limit and upper limit of the lipophilic resin content described above may be employed as appropriate, the more preferred numerical range is preferably 0.01 to 10%, more preferably 0.05 to 8%.

While the trimethylsiloxy silicate and/or polymethyl silsesquioxane content in the entire composition mass is not limited particularly and the preferred lower limit and upper limit of the lipophilic resin content described above may be employed as appropriate, the more preferred numerical range is preferably 0.01 to 10%, more preferably 0.05 to 8%. By combining the both of trimethylsiloxy silicate and polymethyl silsesquioxane, the feel of use without stickiness and the makeup protection effect can be more favorable.

### <2-2. Ethanol>

The ingredient (B) ethanol employed in the present embodiment is not limited particularly as long as it can ordinarily be employed in a cosmetic or a dermal preparation. Ethanol is a water-soluble solvent and an ingredient having volatility at ambient temperature (water-based volatile ingredient). As a result, a refreshing feeling can be obtained upon use by spraying, and the film formation speed, a good feel of use without stickiness and the makeup protection effect can further be favorable.

While the content of the ingredient (B) ethanol is not limited particularly, the preferred lower limit thereof in the entire composition (preferably cosmetic) mass, from the viewpoint of the feel of use without stickiness, the makeup protection effect, the clogging prevention effect and the film formation speed, is preferably 0.1% or more, more preferably 0.5% or more, further preferably 1% or more, more preferably 3% or more, further preferably 5% or more and the preferred upper limit from the viewpoint of clogging prevention is not limited particularly, and is preferably 90% or 80% or 70% or 60% or less, and more preferably 50% or 40% or less from the viewpoint of the fineness of the mist, further preferably 30% or less, more preferably 20% or less, still more preferably 15% or less.

With regard to the ingredient (B) described above to the ingredient (A) in the present invention, the preferred lower limit of the mass content ratio (B)/(A) in the entire composition (preferably cosmetic) mass is not limited particularly and is, from the viewpoint of the film formation speed and the feel of use without stickiness, preferably 0.1 or more, more preferably 0.3 or more, further preferably 0.5 or more, still more preferably 1 or more, more preferably 1.5 or more, more preferably 2 or more, and the preferred upper limit thereof from the viewpoint of the clogging prevention effect on the spray opening or the spray nozzle is preferably 400 or less, more preferably 300 or less, further preferably 200 or less, still more preferably 100 or less, more preferably 50 or less, more preferably 25 or less, more preferably 10 or less, more preferably 5 or less, and a further preferred numerical range is preferably 0.1 to 300, further preferably 1 to 200. As a result, a more favorable makeup protection effect and a more favorable fineness of the mist can readily be achieved together with the feel of use without stickiness, the clogging prevention effect and the film formation speed. The ingredient (A) and the ingredient (B) described above may be a combination of respective preferred examples of the ingredients, and an example may be “ethanol to a lipophilic resin (for example, a silicone-based resin)”. It is also possible that “mass content ratio (B)/(A)” is “used mass ratio (B)/(A)”.

### <2-3. Metal Salt>

The ingredient (C) metal salt (hereinafter referred also as “ingredient (C) metal salt” or “ingredient (C)”) employed in the present embodiment is not limited particularly as long as it can be used ordinarily in a cosmetic or a dermal preparation, and may for example be a metal salt of an inorganic acid and a metal salt of an organic acid, one or more kinds selected from which can be employed. As a result, it is possible to further improve the film formation speed, the feel of use without stickiness, the clogging prevention effect, the makeup protection effect and the fineness of the mist, among which the film formation speed, the feel of use without stickiness and the clogging prevention effect can further be favorable. The ingredient (C) metal salt is preferably a salt of an alkaline metal or a salt of an alkaline earth metal, more preferably a salt of an alkaline metal.

As used in this specification, “a metal salt” is a compound formed from a cation which is a metal ion and an anion, and an acid which releases an anion source electrolytically in water can be exemplified as an inorganic acid or an organic acid. Such a metal salt is preferably an ionic compound. Such an acid is not limited particularly as long as it can be used ordinarily in a cosmetic or a dermal preparation, and may be an inorganic acid and/or an organic acid. Such a metal is not limited particularly as long as it can be used ordinarily in a cosmetic or a dermal preparation, and is preferably one or more kinds selected from an alkaline metal such as lithium, potassium, sodium and the like; an alkaline earth metal such as barium, magnesium, calcium and the like. Among them, a metal salt of an inorganic acid is preferable, an alkaline metal salt or an alkaline earth metal salt is more preferable, and an alkaline metal salt is still more preferable. Among them, a metal salt of an inorganic acid is preferable.

Among the metal salts of inorganic acids, those employed preferably from the viewpoint of the film formation speed and the feel of use without stickiness are preferably one or more kinds selected from a metal chloride, a metal sulfite (for example, sodium pyrosulfite and the like), a metal phosphate (for example, sodium metaphosphate and the like), with an alkaline metal chloride (for example, sodium chloride, potassium chloride, lithium chloride and the like) being more preferred, and sodium chloride being further preferred among them.

Among the metal salts of organic acids, those employed preferably from the viewpoint of the film formation speed and the feel of use without stickiness are preferably one or more kinds selected from a metal citrate, metal salts of vitamin Cs (ascorbic acid and its derivatives (for example, ascorbic phosphate and the like) and the like) and the like, with an alkaline metal citrate and an alkaline metal ascorbic phosphate being more preferred, and sodium citrate and magnesium ascorbic phosphate being further preferred among them.

Among the ingredients (C) metal salts, those employed preferably from the viewpoint of the film formation speed and the feel of use without stickiness are preferably one or more kinds selected from a metal chloride, a metal citrate and a metal salt of vitamin Cs, with a metal chloride being more preferred among them. Compounds exemplified more specifically are one or more kinds selected from, sodium chloride, sodium citrate, magnesium ascorbic phosphate, with sodium chloride being further preferred among them.

The content of the ingredient (C) metal salt in the entire mass of the composition (preferably cosmetic), is not limited particularly, and its preferred lower limit from the viewpoint of the film formation speed, the clogging prevention effect and the fineness of the mist is preferably 0.01% or more, more preferably 0.03% or more, further preferably 0.05% or more, still more preferably 0.1% or more, more preferably 0.3% or more, and its preferred upper limit from the viewpoint of the feel of use without stickiness, the clogging prevention effect and the makeup protection effect is preferably 5% or less, more preferably 4% or less, further preferably 3% or less, still more preferably 2% or less, more preferably 1% or less. The preferred numerical range of the ingredient (D) in the entire mass of the composition is more preferably 0.01 to 5%, still more preferably 0.05 to 3%. Within this range, a more favorable makeup protection effect and a more favorable fineness of the mist can be obtained together with the feel of use without stickiness and the clogging prevention effect, and a more preferable film formation speed can readily be obtained.

With regard to the ingredient (A) described above to the ingredient (C) in the present invention, the preferred lower limit of the mass content ratio (A)/(C) in the composition (preferably cosmetic) is not limited particularly and is, from the viewpoint of the film formation speed and the clogging prevention effect, preferably 0.005 or more, more preferably 0.01 or more, further preferably 0.02 or more, more preferably 0.03 or more, more preferably 0.04 or more, more preferably 0.05 or more, and its preferred upper limit from the viewpoint of the feel of use without stickiness and the makeup protection effect is preferably 25 or less, more preferably 20 or less, further preferably 10 or less, more preferably 8 or less, more preferably 5 or less, more preferably 3 or less, and more preferred numerical range is preferably 0.01 to 25, more preferably 0.02 to 20. As a result, a more favorable makeup protection effect and a more favorable fineness of the mist can be achieved together with the feel of use without stickiness and the clogging prevention effect, and a more preferable film formation speed can readily be obtained. The ingredient (C) and the ingredient (A) described above may be respective preferred examples of the ingredients, and an example may be “sodium chloride to a lipophilic resin (for example, a silicone-based resin)”. It is also possible that “mass content ratio (C)/(A)” is “used mass ratio (C)/(A)”.

### <2-4. Low-Viscosity Non-Volatile Oil Agent>

The ingredient (D) low-viscosity non-volatile oil agent employed in the present embodiment is not limited particularly as long as it can be used ordinarily in a cosmetic or a dermal preparation, and may be any of those derived from a natural oil (animal oil, vegetable oil, microorganism-derived oil and the like), a synthetic oil and the like, which may be in any form such as solid oil, a semi-solid oil, a liquid oil and the like. As a result, it is possible to further improve the film formation speed, the feel of use without stickiness, the clogging prevention effect, the makeup protection effect and the fineness of the mist, among which the film formation speed, the feel of use without stickiness and the clogging prevention effect can further be improved.

As used in this specification, a “non-volatile oil agent” may be a commercial product or available product as a non-volatile oil agent or a non-volatile oil which can be used in a cosmetic or a dermal preparation, and it is preferably an oil agent which does not evaporate at 25° C. under a normal pressure, which may for example be an oil agent having a boiling point under a normal pressure of 260° C. or higher, more preferably a non-volatile oil agent which is in the form of a liquid at 25° C. under a normal pressure.

The low-viscosity non-volatile oil agent is not limited particularly as long as it can be used ordinarily in a cosmetic or a dermal preparation, and may for example be a non-volatile silicone oil such as polyalkyl siloxane (for example, dimethyl polysiloxane and the like), polyalkylaryl siloxane (for example, methylphenyl polysiloxane and the like), polyether siloxane copolymer and the like; hydrocarbon oils such as hydrogenated polyisobutene (also referred to as liquid isoparaffin), liquid paraffin, squalane, and the like; animal and vegetable oils such as mink oil, olive oil, jojoba oil, macadamia nut oil, castor oil, lanolin and the like; an ester oil such as isononanoic acid ester (for example, isotridecyl isononanate, isotridecyl isononanate, octyl isononanate and the like), isopropyl myristate, rosin pentaerythritol ester, glycerin fatty acid ester, triethylhexanoine, cetyl 2-ethylhexanoate, propylene glycol dicaprylate, ethylhexyl palmitate, 2-ethylhexyl paramethoxycinnamate, octocrylene and the like; a higher alcohol such as isostearyl alcohol and the like; a higher fatty acid such as oleic acid, isostearic acid and the like. One or more kinds selected therefrom can be employed, and they have low viscosity.

The ester oil is preferably an oil represented new formula R¹COOR² wherein, R¹ denotes a residue of a straight or branched fatty acid containing 1 to 40 carbon atoms, R² denotes a hydrocarbon chain having 1 to 40 carbon atoms, especially a branched hydrocarbon chain, provided that the sum of the number of carbon atoms in R¹ chain and the number of carbon atoms in R² chain is 10 or more.

Among them, from the viewpoint of the clogging prevention effect, one or more kinds selected from the group consisting of a non-volatile silicone oil such as polyalkyl siloxane (for example, dimethyl polysiloxane and the like), polyalkylaryl siloxane (for example, methylphenyl polysiloxane and the like), polyether siloxanecopolymer and the like; hydrocarbon oils such as hydrogenated polyisobutene, liquid paraffin, squalane and the like; and an ester oil such as isononanoic acid ester (for example, isotridecyl isononanate, isotridecyl isononanate, octyl isononanate and the like) and the like is preferred, with a non-volatile silicone oil being further preferred.

Among the ingredient (D), those employed more preferably from the viewpoint of the clogging prevention effect are one or more kinds selected from the group consisting of a non-volatile silicone oil such as dimethyl polysiloxane or methylphenyl polysiloxane and the like, hydrogenated polyisobutene, isotridecyl isononanate and the like.

In the present embodiment, a commercial product can be employed as the ingredient (D). The commercial product of the ingredient (D) may for example be Silicone KF-96 (5cs), (6cs), (10cs), (20cs), (30cs), (50cs), (100cs), Silicone KF-56 (all manufactured by Shin-Etsu Chemical Co., Ltd.), PARLEAM 6 (manufactured by NOF CORPORATION), SALACOS 913 (manufactured by The Nisshin OilliO Group, Ltd.), UVINUL MC80 (manufactured by BASF SE) and the like, one or more kinds selected from which can be employed.

The viscosity (25° C.) of the low-viscosity non-volatile oil agent is a kinematic viscosity at 25° C. of 300 mm²/s or less, and the upper limit of more preferred kinematic viscosity from the viewpoint of the feel of use without stickiness is preferably 200 mm²/s or less, more preferably 100 mm²/s or less, further preferably 80 mm²/s or less, more preferably 50 mm²/s or less, further preferably 40 mm²/s or less, further preferably 30 mm²/s or less, more preferably 20 mm²/s or less, more preferably 15 mm²/s or less, more preferably 10 mm²/s or less, and the lower limit of a more preferred kinematic viscosity is not limited particularly, and may for example be 1, 2, 3 or 4 mm²/s or more. It is preferable that the viscosity of the respective non-volatile oil employed is within this range of the low viscosity.

The kinematic viscosity (mm²/s) at 25° C. in this specification can be measured using a Brookfield viscometer under the measurement condition described below. The kinematic viscosity (mm²/s) at 25° C. can be calculated based on the viscosity (mPa·s)/density (g/cm³). The viscosity (mPa·s) in this specification can be obtained as a mean value over 1 minutes which is measured using a Brookfield viscometer with Rotor No. 2 at 6 rpm.

The content of the ingredient (D) low-viscosity non-volatile oil agent in the entire mass of the composition (preferably cosmetic) is not limited particularly, and its preferred lower limit from the viewpoint of the makeup protection effect is preferably 0.1% or more, more preferably 0.3% or more, further preferably 0.5% or more, still more preferably 0.8% or more, more preferably 1% or more, more preferably 1.5% or more, more preferably 2% or more, more preferably 3% or more, and its preferred upper limit from the viewpoint of the feel of use without stickiness is preferably 30% or less, more preferably 25% or less, further preferably 20% or less, more preferably 15% or less. The preferred numerical range of the ingredient (A) lipophilic resin content in the entire composition mass is preferably 0.5 to 25%, more preferably 3 to 20%. Within this range, the feel of use without stickiness and the preventive effect on clogging of a spray opening or spray nozzle over time are favorable, and the film forming speed can favorably be adjusted.

With regard to the ingredient (A) to the ingredient (D) in the present embodiment, the mass content ratio (A)/(D) in the composition (preferably cosmetic) is not limited particularly, and its preferred lower limit is preferably 0.005 or more, more preferably 0.01 or more, further preferably 0.03 or more, still more preferably 0.05 or more, more preferably 0.1 or more, more preferably 0.15 or more, and its preferred upper limit is preferably 6 or less, more preferably 3 or less, further preferably 1 or less, more preferably 0.9 or less, more preferably 0.8 or less, more preferably 0.5 or less, with a further preferred numerical range being preferably 0.01 to 6. As a result, more favorable makeup protection effect and more favorable fineness of the mist together with the feel of use without stickiness and preventive effect on clogging of a spray opening or spray nozzle over time are favorable, and the film forming speed can be adjusted favorably. The ingredient (A) and the ingredient (D) may also be respective examples of the preferred ingredients, and may for example be “a lipophilic resin (for example, a silicone-based resin) to a low-viscosity non-volatile oil agent (for example, low viscosity non-volatile silicone oil)”. The “mass content ratio (A)/(D)” may be a “used mass ratio (A)/(D)”.

### <Combined Use of Ingredient (D) and Volatile Oil Agent>

The content of the ingredient (D) low-viscosity non-volatile oil agent is preferable when a volatile oil agent is used in combination. The examples of the compound of the volatile oil agent and the content and the like are described below in Section <2-5-2.>. When using the ingredient (D) and the volatile oil agent in combination, the content in the entire composition (preferably cosmetic) can also be lower than “the content of the ingredient (D) low-viscosity non-volatile oil agent”.

When the ingredient (D) is employed in combination with a volatile oil agent, the preferred lower limit of the content of the ingredient (D) in the entire mass of the composition (preferably cosmetic) from the viewpoint of the film formation speed is preferably 0.1% or more, more preferably 0.5% or more, further preferably 1% or more, still more preferably 2% or more, and the preferred upper limit from the viewpoint of the feel of use without stickiness is preferably 10% or less, more preferably 7% or less, further preferably 5% or less, and the preferred numerical range in the entire mass of the composition is preferably 0.5 to 7%. Within this range, the feel of use without stickiness and the preventive effect on clogging of a spray opening or spray nozzle over time are favorable, and the film forming speed can favorably been adjusted.

In addition, when using the ingredient (D) and a volatile oil agent in combination, the mass content ratio (A)/(D) in the composition (preferably cosmetic) is not limited particularly, and this (A)/(D) mass ratio is preferably elevated, and its preferred lower limit is preferably 0.1 or more, more preferably 0.5 or more, further preferably 1 or more, and its preferred upper limit is preferably 10 or less, more preferably 8 or less, further preferably 6 or less, with a further preferred numerical range being preferably 1 to 6. As a result, more favorable makeup protection effect and more favorable fineness of the mist together with the feel of use without stickiness and preventive effect on clogging of a spray opening or spray nozzle over time are favorable, and the film forming speed can be adjusted favorably. The ingredient (A) and the ingredient (D) may also be respective examples of the preferred ingredients, and the “mass content ratio (A)/(D)” may be a “used mass ratio (A)/(D)”.

When using the ingredient (D) low-viscosity non-volatile oil agent and a volatile oil agent in combination, the mass content ratio of ingredient (D):volatile oil agent is not limited particularly, and is preferably 0.1 to 10:10 to 0.1, more preferably 0.5 to 7:10 to 1, further preferably 1 to 5:10 to 1. The ingredient (D) and the volatile oil agent may also be respective examples of the preferred ingredients, and may for example be “a volatile silicone oil to a low-viscosity non-volatile oil agent (low viscosity non-volatile silicone oil)”. As a result, more favorable makeup protection effect and more favorable fineness of the mist together with the feel of use without stickiness and preventive effect on clogging of a spray opening or spray nozzle over time are favorable, and the film forming speed can be adjusted favorably. The “mass content ratio (D):volatile oil agent” may be a “used mass ratio (D):volatile oil agent”.

### <Combined Use of Ingredient (D) and High-Viscosity Non-Volatile Oil Agent>

As long as the advantageous effects of the present technique are not affected adversely, the ingredient (D) low-viscosity non-volatile oil agent may contain, as an optional ingredient, a non-volatile oil agent having a viscosity which is different from that of the relevant low viscosity non-volatile oil, for example a viscosity which is higher than that of the ingredient (D). With regard to such a high viscosity, the preferred upper limit of the kinematic viscosity at 25° C. is preferably 500 mm²/s or less, more preferably 400 mm²/s or less and the preferred lower limit is preferably 300 mm 2/s or more. The high-viscosity non-volatile oil agent is not limited particularly as long as it can be used ordinarily in a cosmetic or a dermal preparation, and may for example be a non-volatile oil agent having a high-viscosity, and preferably one or more kinds selected from the group consisting of a high-viscosity non-volatile silicone oil, high-viscosity hydrocarbon oils and a high-viscosity ester oil, further preferably a high-viscosity non-volatile silicone oil (dimethyl polysiloxane and the like).

When using the ingredient (D) low-viscosity non-volatile oil agent and a high-viscosity non-volatile oil agent in combination, the content of the high-viscosity non-volatile oil in the entire mass of the composition (preferably cosmetic) is preferably 10% or less, more preferably 5% or less, further preferably 3% or less, more preferably 1% or less. The content of the high-viscosity non-volatile oil agent in the entire amount of the non-volatile oil agent (the low viscosity non-volatile oil agent described above and a non-volatile oil agent having a viscosity different therefrom) is preferably not an amount of 30 mass % or more, more preferably substantially zero (not 1 or 0.5 mass % or more). When using the ingredient (D) low-viscosity non-volatile oil agent and a high-viscosity non-volatile oil agent in combination, the high-viscosity non-volatile oil agent is contained preferably in a ratio giving the range of “the viscosity of the low-viscosity non-volatile oil agent (25° C.)” described above when mixing the low viscosity non-volatile oil agent and a non-volatile oil agent having a higher viscosity.

### <2-5. Optional Ingredients>

The multi-layer makeup protective cosmetic of the present embodiment can contain, in addition to (A) to (D) and ingredients described above, those ingredient employed in an ordinary cosmetic or dermal preparation if necessary as long as the advantageous effects of the present technique are not affected adversely. Those which may be exemplified are water, a volatile oil agent, a non-volatile oil agent, a monohydric lower alcohol, a surfactant (emulsifier), an oil-based ingredient, a water-based ingredient, a powder, a wax, a moisturizer, a thickening agent, a preservative, a UV absorber, a pH adjusting agent, a fragrance, a medical ingredient, a propellant and the like. One or more kinds selected therefrom can be employed.

### <2-5-1. Water>

In the present embodiment, it is preferable that water is further contained. The present embodiment can provide a multi-layer makeup protective cosmetic containing ingredients (A) to (D) and water as a preferred embodiment. As a result, a more favorable makeup protection effect and a more favorable fineness of the mist can be obtained. While water is not limited particularly as long as it can be used ordinarily in a cosmetic or a dermal preparation, those which can be employed in addition to a purified water are an ion exchange water, a seawater, a deep sea water, or a steam-distilled plant water, one or more kinds selected from which can be employed.

In the present embodiment, the water content is not limited particularly, its preferred lower limit in the entire mass of the composition (preferably cosmetic), is preferably 1% or more, more preferably 3% or more, further preferably 5% or more, still more preferably 10% or more, more preferably 15% or more, more preferably 20% or more, more preferably 25% or more, more preferably 30% or more, more preferably 40% or more, more preferably 45% or more, more preferably 50% or more, more preferably 55% or more, more preferably 60% or more, more preferably 65% or more, and its preferred upper limit is preferably 98% or less, more preferably 95% or less, further preferably 93% or less, still more preferably 90% or less, more preferably 87% or less, more preferably 85% or less, more preferably 80% or less, more preferably 75% or less, more preferably 70% or less. The preferred numerical range of the water in the entire mass of the composition is preferably 30 to 95%, more preferably 50 to 85%. As a result, a more favorable makeup protection effect and a more favorable fineness of the mist together with a preferable film forming speed and a good feel of use without stickiness can readily be obtained, and prevention of clogging of a spray opening or spray nozzle occurring over time can readily be achieved.

### <2-5-2. Volatile Oil Agent>

In the present embodiment, it is preferable that a volatile oil agent is further contained. The “volatile oil agent” as used in this specification is not limited particularly as long as it is in a liquid form and has a volatility at 20° C. under a normal pressure, regardless of the viscosity or the origin. By using a volatile oil agent, the ingredient (A) lipophilic resin can be dissolved and contained stably in a composition. The present embodiment can also provide, as a preferable embodiment, a multi-layer makeup protective cosmetic containing the ingredients (A) to (D) and a volatile oil agent. As a result, a more favorable makeup protection effect and a more favorable fineness of the mist can be achieved together with the feel of use without stickiness and the clogging prevention effect, and a more preferable film formation speed can readily be obtained.

The volatile oil agent is not limited particularly as long as it can ordinarily employed in a cosmetic or a dermal preparation, and may for example be a silicone oil, a hydrocarbon oil, an ester oil and the like, one or more kinds from which can be selected for use. Those preferred among them are a silicone oil and/or a hydrocarbon oil.

Examples of the volatile oil agent may more specifically be, but not limited to, a silicone oil such as low-molecular-weight methylpolysiloxanes, decamethyl cyclopentasiloxane, octamethyl cyclotetrasiloxane, dodecamethyl cyclohexasiloxane, methyl trimethicone, decamethyl tetrasiloxane, and ethyl trisiloxane; a hydrocarbon oil such as light liquid isoparaffin, isododecane, and isohexadecane, and the like, which are volatile. One or more kinds thereof can be used.

Commercially available products of volatile oil agents described above may for example be Isopar H (manufactured by Esso Chemical Co., Ltd.), Isododecane (manufactured by Bayer AG), Isohexadecane (manufactured by Uniqema), and IP Solvent 1620MU, IP Solvent 2028MU, and IP Solvent 2835 (all manufactured by Idemitsu Kosan Co., Ltd.) as light liquid isoparaffin; TSF405 (manufactured by Toshiba Silicone Co., Ltd.), SH245 and DC345 (manufactured by Dow Corning Toray Co., Ltd.), and KF-995 (manufactured by Shin-Etsu Chemical Co., Ltd.) as decamethyl cyclopentasiloxane; Silicone TMF-1.5 (manufactured by Shin-Etsu Chemical Co., Ltd.) as methyl trimethicone; KF-96L-2CS (manufactured by Shin-Etsu Chemical Co., Ltd.) as low-molecular-weight methylpolysiloxane; KF-96L-1.5CS (manufactured by Shin-Etsu Chemical Co., Ltd.) as decamethyl tetrasiloxane; SILSOFT ETS (manufactured by Momentive Performance Materials Inc.) as ethyl trisiloxane; and the like. One or more kinds thereof can be selected and used.

Among the volatile oil agents described above, a volatile silicone oil (preferably having a kinematic viscosity of 2 mm²/s or less at 25° C.) is preferred from the viewpoint of no stickiness, and more specifically, one or more kinds selected from decamethyl cyclopentasiloxane, methyl trimethicone, volatile dimethyl polysiloxane (preferably dimethyl polysiloxane having a kinematic viscosity of 2 mm²/s or less at 25° C.) and the like are preferred.

The content of the volatile oil agent in the present embodiment is not limited particularly, and its preferred lower limit in the entire mass of the composition (preferably cosmetic), from the viewpoint of the film formation speed, is preferably 1% or more, more preferably 3% or more, further preferably 4% or more, still more preferably 5% or more, and its preferred upper limit from the viewpoint of the film formation speed is preferably 40% or less, more preferably 30% or less, further preferably 25% or less, further preferably 20% or less, more preferably 15% or less. As a result, a fineness of the mist when used by spraying, an excellent makeup protection effect and a more preferable film formation speed can be achieved.

The preferred numerical range of this volatile oil agent in the entire mass of the composition is preferably 1 to 20%, more preferably 5 to 15%. Within this range, a multi-layer makeup protective cosmetic having a favorable film forming speed when used and a good feel of use with less stickiness can be obtained.

### <2-5-3. Other Ingredients>

The water-soluble polymer is not limited particularly as long as it can be used ordinarily in a cosmetic or a dermal preparation, and may for example be guar gum, sclerotium gum, gellan gum, pectin, agar, sodium chondroitin sulfate, hyaluronic acid, gum arabic, sodium alginate, carrageenan, xanthan gum, locust bean gum, methyl cellulose, hydroxyethyl cellulose, carboxymethyl cellulose, carboxyvinyl polymers, alkyl-modified carboxyvinyl polymers, polyvinyl alcohol, polyvinyl pyrrolidone, (sodium acrylate/sodium acryloyldimethyltaurine) copolymers, (PEG-240/decyltetradeceth-20/HDI) copolymers, and the like, and one or more kinds selected therefrom can be used.

The water-based ingredient other than the ingredient (B) describe above is not limited particularly as long as it can be used ordinarily in a cosmetic or a dermal preparation, and may for example be glycerols such as glycerin, diglycerin, and polyglycerin; sugar alcohols such as sorbitol, maltitol, and glucose; monohydric or polyhydric lower alcohols such as 1,3-butylene glycol and the like, and one or more kinds selected therefrom can be used. In addition, a water-based ingredient preferably can function also as a solvent, and more preferably is water-soluble and forms a water layer. The lower alcohol preferably has 1 to 4 carbon atoms, and is preferably 1,3-butylene glycol.

The content of the water-based ingredient in the present embodiment is not limited particularly, and its preferred lower limit in the entire mass of the composition (preferably cosmetic) is preferably 1% or more, more preferably 3% or more, further preferably 5% or more, and its preferred upper limit is preferably 30% or less, more preferably 25% or less, further preferably 20% or less, still more preferably 15% or less.

The oil-based ingredient is not limited particularly as long as it can be used ordinarily in a cosmetic or a dermal preparation, and may for example be those derived from animal oils, vegetable oils, synthetic oils and the like regardless of the forms such as solid oils, semi-solid oils, liquid oils and the like, such as hydrocarbon oils, fats and oils, hydrogenated oils, ester oils, fatty acids, non-volatile silicone oils and the like. More specifically, hydrocarbon oils such as liquid paraffin, squalane, polyisobutylene, and polybutene, oils and fats such as olive oil, castor oil, and macadamia nut oil, esters such as cetyl isooctanate, isopropyl myristate, isopropyl palmitate, octyldodecyl myristate, and polyglyceryl diisostearate, fatty acids such as stearic acid, lauric acid, myristic acid, behenic acid, isostearic acid, and oleic acid, non-volatile silicone oils such as dimethylpolysiloxane and methylphenylpolysiloxane, essential oils, fragrances, and the like can be mentioned. One or more kinds selected therefrom can be used.

The powder is not particularly limited as long as it can be used in an ordinary cosmetic, and may for example be an inorganic powder such as titanium oxide, zinc oxide, cerium oxide, aluminum oxide, silicic anhydride, calcium carbonate, chromium oxide, chromium hydroxide, iron blue, ultramarine blue, iron oxide, carbon black, mica, synthetic gold mica, sericite, talc, kaolin, barium sulfate, and boron nitride, nylon, polymethyl methacrylate, polyethylene, polypropylene, polystyrene, silicone resin powders, cellulose and derivatives thereof, urethane, silk powders, crystalline cellulose, N-acyl lysine, and the like, an organic tar-based pigment such as Red No. 201, Red No. 202, Red No. 228, Orange No. 203, Blue No. 404, Yellow No. 401, Red No. 3, Red No. 104, Red No. 106, Orange No. 205, Yellow No. 4, Yellow No. 5, Green No. 3, Blue No. 1, Purple No. 401, and Purple No. 201, as well as a pigment powder of such a lake pigment, a composite powder such as mica titanium, titanium oxide-coated mica titanium, zinc oxide-coated mica titanium, titanium oxide-coated glass powders, and carmine-coated mica titanium, a laminated film powder such as a polyethylene terephthalate/aluminum/epoxy laminate powder and a polyethylene terephthalate/polymethyl methacrylate laminate powder, and a metal powder such as an aluminum powder, gold, and silver. Such a powder may be surface-treated using one or more kinds of a fluorine-based compound, a silicone-based compound, a metal soap, lecithin, hydrogenated lecithin, collagen, an amino acid, a hydrocarbon, a higher fatty acid, a higher alcohol, an ester, a grease, a wax, a surfactants and the like. One or more kinds selected therefrom can be used. When such a powder is contained, it is also possible to provide the multi-layer structure of the makeup protection cosmetic with three or more layers to the extent the advantages of the present invention are not impaired.

The powder content in this embodiment is not particularly limited, and its preferred lower limit in the entire mass of the composition (preferably cosmetic) is preferably 0.5% or more, and more preferably 1% or more, and its preferred upper limit is preferably 10% or less, more preferably 5% or less, and still more preferably 3% or less.

The antioxidant is not limited particularly and may for example be, but not limited to, α-tocopherol, ascorbic acid, and the like; beauty ingredients may for example be vitamins, anti-inflammatories, crude drugs, and the like; and preservatives may be paraoxybenzoic acid esters, phenoxyethanol, alkanediol, chlorphenesin and the like, and one or more kinds thereof can be used.

The pH adjuster is not limited particularly, and may for example be, lactic acid, citric acid, glycolic acid, succinic acid, tartaric acid, malic acid, ammonium hydrogen carbonate, triethanolamine, isopropanolamine, a basic amino acid and the like; and a refreshing agent is not limited particularly may for example be, but not limited to, L-menthol, camphor and the like, and one or more kinds thereof can be suitably selected and used.

The emulsifier is not limited particularly as long as it is used ordinarily in a cosmetic or a dermal preparation. The multi-layer makeup protective cosmetic in the present embodiment preferably contains substantially no emulsifier from the viewpoint of a favorable fineness of the mist and a favorable aesthetic quality of the multilayer. Even when an emulsifier is contained, the emulsifier quantity is preferably small from the viewpoint of an improved lasting of makeup. Containing substantially no emulsifier means that the emulsifier content in the entire quantity of the composition (preferably cosmetic) is less than 0.25%, preferably less than 0.1%, more preferably less than 0.05%, most preferably zero. In other words it is preferable from the viewpoint of emulsifier-free or low-emulsifier that the emulsifier is not contained in the entire quantity of the composition at 0.25% or more, preferably 0.1% or more, more preferably 0.05% or more.

The present embodiment may be an aerosol-type makeup protective cosmetic and the like, and the ingredients or compositions and the like used in the makeup protective cosmetic described above are used as a stock solution, and an aerosol-type cosmetic consisting of such a stock solution and a propellant may be provided. The present embodiment gives an aerosol product employing a known method for producing an aerosol product or a known method for charging an aerosol to an aerosol container (for example, an accumulator spray container, aerosol metal container and the like). The mass ratio of the stock solution and the propellant described above is not limited particularly, and may for example be stock solution 10 to 50: propellant 90 to 50. The propellant is not limited particularly as long as it is used ordinarily in a cosmetic or a dermal preparation, and may contain one or more kinds selected from a liquefied gas, carbon dioxide, nitrogen, argon and the like. It is preferred to use at least a liquefied gas as a propellant. The liquefied gas is not limited particularly and may for example be a hydrocarbon having 2 to 5 carbon atoms and ethers and the like. The hydrocarbon having 2 to 5 carbon atoms may for example be ethane, propane (hereinafter also referred to as “LPG”), n-butane, isobutane, n-pentane, isopentane, neopentane and the like. The ethers may for example be dimethyl ether (hereinafter also referred to as “DME”), diethyl ether and the like. One or more kinds thereof may be selected and used as appropriate.

### <Multi-Layer Makeup Protective Cosmetic of Present Embodiment and Production Method>

The method for producing a multi-layer makeup protective cosmetic of the present embodiment is not limited particularly, and production is possible by a known method, such as charging ingredients (A) to (D) and water, other ingredients as appropriate into a container (for example, a non-aerosol container such as a container with a dispenser, a container with a spray nozzle, a spray container, a finger spray container, a mist spray container and the like; an aerosol container and the like). It is also possible that the ingredients employed in the present embodiment are mixed and then charged, and in such a case the mixing may be conducted with heating (for example 80 to 90° C.) as appropriate. It is also possible that the ingredient in the present embodiment is charged into a plurality of containers, such as a container for a water layer, a container for an oil layer, and a container for a powder and provided as a multi-component combination product such as a two-component or three-component product. Also in the present embodiment, a combination with apparatus such as a container, applicator and the like can be mentioned. Such a combination may be a set or a kit. In case of a multi-component product, a user may mix the water layer ingredient and the oil layer ingredient, the powder ingredient and the like in a desired ratio, and then spray the product. While an aerosol form using a propellant may be used in the present embodiment, it is preferable to use a spray container using no propellant from the viewpoint of easy spray onto the face.

The multi-layer makeup protective cosmetic of the present embodiment is preferably used by spraying onto the face with base makeup such as foundation, or makeup such as eye shadow or blush, and a container with a spray nozzle can be used. A spray container having a spray nozzle with a spray aperture (diameter) of approximately φ0.1 to 0.6 mm (preferably φ0.2 to 0.5 mm) may be used, and an aperture of φ0.3 mm may be used for spray evaluation. The cosmetic is for use by spraying, and thus is preferably a liquid cosmetic.

As shown below in [Examples], the makeup protective cosmetic of the present embodiment has a multi-layer structure having at least two layers including an upper oil layer and a lower water layer, and can also contain a powder or the like to form three or more layers. In a preferable form of the multi-layer makeup protective cosmetic of the present embodiment, it can favorably be dispersed by mixing just before use by spraying, and such a dispersed state can be maintained for a certain period of time, while it returns to the multi-layer structure or undergoes phase separation when being left for a certain period of time.

Since a multi-layer makeup protective cosmetic of the present embodiment is in a favorable dispersion state for a certain period after mixing, a mist-like fine particles can be sprayed. In addition, the present embodiment exhibits, even in a multilayer form, excellent spraying performance and excellent film formation, which are attributable to incorporation of a lipophilic resin and combination of ingredients (A) to (D) and water. In addition, since the present embodiment gives a fine spray particle even when no emulsifier is incorporated, it can advantageously provide an emulsifier-free or a low-emulsifier makeup protective cosmetic.

Moreover, according to the present embodiment, because of the combination of the ingredients (A) to (D), a film with high water resistance attributable to the lipophilic resin and sebum resistance can be formed on the applied makeup, offering an excellent makeup protection effect with excellent durability. Furthermore, according to the present embodiment, mist-like fine particles can be sprayed all over the face, and a film having high water resistance and water repellency can be formed all over the face.

The viscosity (mPa·s) of the composition (preferably cosmetic) of the present embodiment is preferably 1,500 mPa·s or less in 1-minute average measurement at 25° C. using a Brookfield viscometer with Rotor No. 2 at 6 rpm, and the viscosity is more preferably 1,000 mPa's or less which enables the fineness of the mist to be excellent.

In the aforementioned description of the examples of the multilayer skin protective composition, makeup protective cosmetic and the like according to the present embodiment, the descriptions with regard to the respective technical characteristics, the respective constitutions, the respective definitions, the respective terms, the respective processing methods, the respective means and the like of the ingredients such as the ingredient (A) lipophilic resin, the ingredient (B) ethanol, the ingredient (C) metal salt and the ingredient (D) low-viscosity non-volatile oil agent, water, a volatile oil agent and the like, and examples of these compounds, their contents or mass content ratios, used quantities or used mass ratios, which are the same to or overlapping the context of the aforementioned description (for example “1.” and the like) and the context of the following description (for example “3.” to “4.” and the like) and the like, are omitted as appropriate, and the description of “1.” to “4.” and the like may apply to the respective embodiments and may be adopted in the respective embodiments.

### 3. Another Aspect of Present Embodiment

In the aforementioned description of examples of the multilayer skin protective composition, makeup protective cosmetic and the like according to another aspect of the present embodiment described above, the descriptions with regard to the respective technical characteristics, the respective constitutions, the respective definitions, the respective terms, the respective processing methods, the respective means and the like of the ingredients such as the ingredient (A) lipophilic resin, the ingredient (B) ethanol, the ingredient (C) metal salt and the ingredient (D) low-viscosity non-volatile oil agent, water, a volatile oil agent and the like, and examples of these compounds, their contents or mass content ratios, used quantities or used mass ratios, which are the same to or overlapping the context of the aforementioned description (for example “1.” to “2.” and the like) and the context of the following description (for example “4.” and the like) and the like, are omitted as appropriate, and the description of “1.” to “4.” and the like may apply to the respective embodiments and may be adopted in the respective embodiments.

In another aspect of the present embodiment (hereinafter also referred to as “the present second embodiment”), by using an ingredient (B) ethanol, an ingredient (C) metal salt, an ingredient (D) low-viscosity non-volatile oil agent in a multilayer skin protective composition (preferably a multi-layer makeup protective cosmetic) containing an ingredient (A) lipophilic resin, it becomes possible to impart the aforementioned composition with a preventive effect on clogging over time, together with a feel of use without stickiness, a makeup protection effect and a fineness of the mist, and also possible to make film formation favorable.

In the present second embodiment, the aforementioned imparting method may be a method for increasing or improving the preventive effect on clogging over time and the feel of use without stickiness when compared to the aforementioned composition. The prevention of clogging of the spray opening or spray nozzle over time means precaution, remedy, improvement, inhibition and the like against the clogging of the inside or the tip of the spray opening or spray nozzle by discharging or spraying, from the spray opening or spray nozzle, the composition or the cosmetic and the like contained in the container over time and may be a favorable turn or maintenance of the condition; prevention or retardation of the exacerbation; reversal, prevention or retardation of advancement. The “content in the entire mass of the composition” and “mass content ratio” in the ingredient (A) to ingredient (D) and optional ingredients described in “2.” may be “used amount” and “used mass ratio”.

### 4. The Present Technique can Adopt the Following Technical Characteristics, Constitutions or Other Aspects.

[1] A multilayer skin protective composition or a multi-layer makeup protective cosmetic comprising an ingredient (A) lipophilic resin, an ingredient (B) ethanol, an ingredient (C) metal salt and an ingredient (D) low-viscosity non-volatile oil agent. This ingredient (D) preferably has a kinematic viscosity at 25° C. of less than 300 mm²/s.
[2] The composition or cosmetic according the aforementioned [1] which is used by spraying, more preferably spraying over face or makeup.
[3] The composition or cosmetic according the aforementioned [1] or [2] wherein the ingredient (A) lipophilic resin is a silicone-based resin.
[4] The composition or cosmetic according any one of the aforementioned [1] to [3] wherein the ingredient (C) is a salt of an alkaline metal or a salt of an alkaline earth metal, or a metal salt of an inorganic acid.
[5] The composition or cosmetic according any one of the aforementioned [1] to [4] wherein the ingredient (D) is one or more kinds selected from the group consisting of a silicone oil, an ester oil and a hydrocarbon oil.
[6] The composition or cosmetic according any one of the aforementioned [1] to [5] wherein the mass content ratio (B)/(A) of the ingredient (B) to the ingredient (A) is 1 to 200.
[7] The composition or cosmetic according any one of the aforementioned [1] to [6] wherein the mass content ratio (C)/(A) of the ingredient (C) to the ingredient (A) is 0.02 to 20.
[8] The composition or cosmetic according any one of the aforementioned [1] to [7] wherein the mass content ratio (A)/(D) of the ingredient (A) to the ingredient (D) is 0.01 to 6 or 0.9 or less.
[9] The composition or cosmetic according any one of the aforementioned [1] to [8] wherein water and/or a volatile oil agent is further contained.
[10] A method for imparting a clogging prevention effect to or improving a clogging of a multilayer skin protective composition or a multi-layer makeup protective cosmetic containing an ingredient (A) lipophilic resin by using an ingredient (B) ethanol, an ingredient (C) metal salt and an ingredient (D) low-viscosity non-volatile oil agent in the aforementioned composition or the aforementioned cosmetic. More preferably water and/or a volatile oil agent is employed.
[11] A skin protection method or a makeup method, a re-makeup method, a makeup protection method by using a multilayer skin protective composition or multi-layer makeup protective cosmetic according any one of the aforementioned [1] to [9] over the makeup and/or skin and/or facemask and the like, or at the time of makeup (before, during or after makeup step), or at a time when a certain time (several hours (a few or several; 2 to 6, 2 to 3, 4 to 6) elapsed after makeup.

### EXAMPLES

Hereinafter, the present technique is further detailed based on Examples and the like. The following Examples and the like show the representative examples of the present technique, by which the scope of the present technique is not interpreted narrowly.

### Multi-Layer Makeup Protective Cosmetic: Examples 1 to 28 and Comparatives 1 to 5, Reference Example 1

Multi-layer makeup protection products having the compositions shown in Tables 1 to 3 were prepared by the following production method, and evaluated and judged according to the evaluation methods and judgment criteria shown below, and the results are also shown in the tables.

TABLE 1

| | No. | Name of ingredient | Example 1 | Example 2 | Example 3 | Example 4 | Example 5 | Example 6 | Example 7 |
|---|---|---|---|---|---|---|---|---|---|
| (A) | 1 | Trimethylsiloxysilicic acid | 3 | 8 | 0.05 | — | — | — | — |
| | 2 | Polymethylsilsesquioxane | — | — | — | 3 | — | — | — |
| | 3 | (Dimethicone/vinyltrimethylsiloxysilicic acid) cross- | — | — | — | — | 3 | — | — |
| | | polymer | | | | | | | |
| | 4 | (Acrylates/dimethicone) copolymer | — | — | — | — | — | 3 | — |
| | 5 | Trifluoroalkyldimethyl trimethylsiloxysilicic acid | — | — | — | — | — | — | 3 |
| | 6 | (Trimethylsiloxysilicic acid/dimethiconol) cross- | — | — | — | — | — | — | — |
| | | polymer | | | | | | | |
| | 7 | (Dimethicone/vinyldimethicone) cross-polymer | — | — | — | — | — | — | — |
| | 8 | (Styrene/acrylates) copolymer | — | — | — | — | — | — | — |
| | 9 | (Cyclohexyl methacrylate/ethylhexyl metacrylate) | — | — | — | — | — | — | — |
| | | copolymer | | | | | | | |
| | 10 | Dextrin isostearate | — | — | — | — | — | — | — |
| | 11 | PVP | — | — | — | — | — | — | — |
| (B) | 12 | Ethanol | 10 | 10 | 10 | 10 | 10 | 10 | 10 |
| | 13 | Glycerin | — | — | — | — | — | — | — |
| (C) | 14 | Sodium chloride | 1 | 1 | 1 | 1 | 1 | 1 | 1 |
| | 15 | Sodium citrate pKa1 = 3.09 pKa2 = 4.75 pKa3 = 6.41 | — | — | — | — | — | — | — |
| | 16 | Ascorbyl phosphate Mg | — | — | — | — | — | — | — |
| (D) | 17 | Dimethyl polysiloxane (25° C., 6 mm²/s) | 10 | 10 | 10 | 10 | 10 | 10 | 10 |
| | 18 | Dimethyl polysiloxane (2° C., 20 mm²/s) | — | — | — | — | — | — | — |
| | 19 | Hydrogenated polyisobutene (25° C., 6 mm²/s) | — | — | — | — | — | — | — |
| | 20 | Isotridecyl isononanate (25° C., 11 mm²/s) | | | — | — | — | — | — |
| | 21 | Dimethyl polysiloxane (25° C., 100 mm²/s) | — | — | — | — | — | — | — |
| | 22 | Dimethyl polysiloxane (25° C., 300 mm²/s) | | | — | — | — | — | — |
| | 23 | Dimethyl polysiloxane (25° C., 1.5 mm²/s) (Volatile) | — | — | — | — | — | — | — |
| | 24 | Water | Remainder | Remainder | Remainder | Remainder | Remainder | Remainder | Remainder |
| | 25 | Phenoxyethanol | 0.3 | 0.3 | 0.3 | 0.3 | 0.3 | 0.3 | 0.3 |
| | | B/A | 3.3 | 1.3 | 200.0 | 3.3 | 3.3 | 3.3 | 3.3 |
| | | C/A | 0.33 | 0.13 | 20.00 | 0.33 | 0.33 | 0.33 | 0.33 |
| | | A/D | 0.30 | 0.80 | 0.01 | 0.30 | 0.30 | 0.30 | 0.30 |
| | | Evaluation | | | | | | | |
| | | Film formation speed | ⊚ | ◯ | ◯ | ◯ | ⊚ | ⊚ | ◯ |
| | | Feel of use without stickiness | ◯ | ◯ | ⊚ | ⊚ | ◯ | ◯ | ◯ |
| | | Clogging prevention effect | ⊚ | ◯ | ⊚ | ⊚ | ◯ | ⊚ | ⊚ |
| | | Makeup protection | ⊚ | ⊚ | ◯ | ⊚ | ⊚ | ◯ | ⊚ |
| | | Fineness of mist | ⊚ | ◯ | ⊚ | ⊚ | ⊚ | ⊚ | ⊚ |

| | No. | Name of ingredient | Example 8 | Example 9 | Example 10 | Example 11 | Example 12 | Example 13 |
|---|---|---|---|---|---|---|---|---|
| (A) | 1 | Trimethylsiloxysilicic acid | — | — | — | — | — | 1.5 |
| | 2 | Polymethylsilsesquioxane | — | — | — | — | — | 1.5 |
| | 3 | (Dimethicone/vinyltrimethylsiloxysilicic acid) cross- | — | — | — | — | — | — |
| | | polymer | | | | | | |
| | 4 | (Acrylates/dimethicone) copolymer | — | — | — | — | — | — |
| | 5 | Trifluoroalkyldimethyl trimethylsiloxysilicic acid | — | — | — | — | — | — |
| | 6 | (Trimethylsiloxysilicic acid/dimethiconol) cross- | 3 | — | — | — | — | — |
| | | polymer | | | | | | |
| | 7 | (Dimethicone/vinyldimethicone) cross-polymer | — | 3 | — | — | — | — |
| | 8 | (Styrene/acrylates) copolymer | — | — | 3 | — | — | — |
| | 9 | (Cyclohexyl methacrylate/ethylhexyl metacrylate) | — | — | — | 3 | — | — |
| | | copolymer | | | | | | |
| | 10 | Dextrin isostearate | — | — | — | — | 3 | — |
| | 11 | PVP | — | — | — | — | — | — |
| (B) | 12 | Ethanol | 10 | 10 | 10 | 10 | 10 | 10 |
| | 13 | Glycerin | — | — | — | — | — | — |
| (C) | 14 | Sodium chloride | 1 | 1 | 1 | 1 | 1 | 1 |
| | 15 | Sodium citrate pKa1 = 3.09 pKa2 = 4.75 pKa3 = 6.41 | — | — | — | — | — | — |
| | 16 | Ascorbyl phosphate Mg | — | — | — | — | — | — |
| (D) | 17 | Dimethyl polysiloxane (25° C., 6 mm²/s) | 10 | 10 | 10 | 10 | 10 | 10 |
| | 18 | Dimethyl polysiloxane (2° C., 20 mm²/s) | — | — | — | — | — | — |
| | 19 | Hydrogenated polyisobutene (25° C., 6 mm²/s) | — | — | — | — | — | — |
| | 20 | Isotridecyl isononanate (25° C., 11 mm²/s) | — | — | — | — | — | — |
| | 21 | Dimethyl polysiloxane (25° C., 100 mm²/s) | — | — | — | — | — | — |
| | 22 | Dimethyl polysiloxane (25° C., 300 mm²/s) | — | — | — | — | — | — |
| | 23 | Dimethyl polysiloxane (25° C., 1.5 mm²/s) (Volatile) | — | — | — | — | — | — |
| | 24 | Water | Remainder | Remainder | Remainder | Remainder | Remainder | Remainder |
| | 25 | Phenoxyethanol | 0.3 | 0.3 | 0.3 | 0.3 | 0.3 | 0.3 |
| | | B/A | 3.3 | 3.3 | 3.3 | 3.3 | 3.3 | 3.3 |
| | | C/A | 0.33 | 0.33 | 0.33 | 0.33 | 0.33 | 0.33 |
| | | A/D | 0.30 | 0.30 | 0.30 | 0.30 | 0.30 | 0.30 |
| | | Evaluation | | | | | | |
| | | Film formation speed | ◯ | ◯ | ◯ | ◯ | ◯ | ⊚ |
| | | Feel of use without stickiness | ◯ | ◯ | ◯ | ◯ | ◯ | ⊚ |
| | | Clogging prevention effect | ⊚ | ⊚ | ◯ | ◯ | ◯ | ⊚ |
| | | Makeup protection | ⊚ | ◯ | ◯ | ◯ | ◯ | ⊚ |
| | | Fineness of mist | ⊚ | ⊚ | ⊚ | ⊚ | ⊚ | ⊚ |

TABLE 2

| | | | Example | Example | Example | Example | Example | Example | Example |
|---|---|---|---|---|---|---|---|---|---|
| | No. | Name of ingredient | 14 | 15 | 16 | 17 | 18 | 19 | 20 |
| (A) | 1 | Trimethylsiloxysilicic acid | 3 | 3 | 3 | 3 | 3 | 3 | 3 |
| | 2 | Polymethylsilsesquioxane | — | — | — | — | — | — | — |
| | 3 | (Dimethicone/vinyltrimethylsiloxysilicic acid) cross- | — | — | — | — | — | — | — |
| | | polymer | | | | | | | |
| | 4 | (Acrylates/dimethicone) copolymer | — | — | — | — | — | — | — |
| | 5 | Trifluoroalkyldimethyl trimethylsiloxysilicic acid | — | — | — | — | — | — | — |
| | 6 | (Trimethylsiloxysilicic acid/dimethiconol) cross- | — | — | — | — | — | — | — |
| | | polymer | | | | | | | |
| | 7 | (Dimethicone/vinyldimethicone) cross-polymer | — | — | — | — | — | — | — |
| | 8 | (Styrene/acrylates) copolymer | — | — | — | — | — | — | — |
| | 9 | (Cyclohexyl methacrylate/ethylhexyl metacrylate) | — | — | — | — | — | — | — |
| | | copolymer | | | | | | | |
| | 10 | Dextrin isostearate | — | — | — | — | — | — | — |
| | 11 | PVP | — | — | — | — | — | — | — |
| (B) | 12 | Ethanol | 30 | 1 | 10 | 10 | 10 | 10 | 10 |
| | 13 | Glycerin | — | — | — | — | — | — | — |
| (C) | 14 | Sodium chloride | 1 | 1 | 3 | 0.05 | — | — | 1 |
| | 15 | Sodium citrate pKa1 = 3.09 pKa2 = 4.75 pKa3 = 6.41 | — | — | — | — | 1 | — | — |
| | 16 | Ascorbyl phosphate Mg | — | — | — | — | — | 1 | — |
| (D) | 17 | Dimethyl polysiloxane (25° C., 6 mm²/s) | 10 | 10 | 10 | 10 | 10 | 10 | 20 |
| | 18 | Dimethyl polysiloxane (2° C., 20 mm²/s) | — | — | — | — | — | — | — |
| | 19 | Hydrogenated polyisobutene (25° C., 6 mm²/s) | — | — | — | — | — | — | — |
| | 20 | Isotridecyl isononanate (25° C., 11 mm²/s) | — | — | — | — | — | — | — |
| | 21 | Dimethyl polysiloxane (25° C., 100 mm²/s) | — | — | — | — | — | — | — |
| | 22 | Dimethyl polysiloxane (25° C., 300 mm²/s) | — | — | — | — | — | — | — |
| | 23 | Dimethyl polysiloxane (25° C., 1.5 mm²/s) (Volatile) | — | — | — | — | — | — | — |
| | 24 | Water | Remainder | Remainder | Remainder | Remainder | Remainder | Remainder | Remainder |
| | 25 | Phenoxyethanol | 0.3 | 0.3 | 0.3 | 0.3 | 0.3 | 0.3 | 0.3 |
| | | B/A | 10.0 | 0.3 | 3.3 | 3.3 | 3.3 | 3.3 | 3.3 |
| | | C/A | 0.33 | 0.33 | 1.00 | 0.02 | 0.33 | 0.33 | 0.33 |
| | | A/D | 0.30 | 0.30 | 0.30 | 0.30 | 0.30 | 0.30 | 0.15 |
| | | Evaluation | | | | | | | |
| | | Film formation speed | ⊚ | ◯ | ⊚ | ◯ | ◯ | ◯ | ◯ |
| | | Feel of use without stickiness | ⊚ | ◯ | ◯ | ⊚ | ◯ | ◯ | ◯ |
| | | Clogging prevention effect | ◯ | ⊚ | ⊚ | ⊚ | ⊚ | ⊚ | ⊚ |
| | | Makeup protection | ⊚ | ◯ | ◯ | ⊚ | ⊚ | ⊚ | ◯ |
| | | Fineness of mist | ◯ | ⊚ | ⊚ | ◯ | ⊚ | ⊚ | ⊚ |

| | | | Example | Example | Example | Example | Example | Example |
|---|---|---|---|---|---|---|---|---|
| | No. | Name of ingredient | 21 | 22 | 23 | 24 | 25 | 26 |
| (A) | 1 | Trimethylsiloxysilicic acid | 3 | 3 | 3 | 3 | 3 | 3 |
| | 2 | Polymethylsilsesquioxane | — | — | — | — | — | — |
| | 3 | (Dimethicone/vinyltrimethylsiloxysilicic acid) cross- | — | — | — | — | — | — |
| | | polymer | | | | | | |
| | 4 | (Acrylates/dimethicone) copolymer | — | — | — | — | — | — |
| | 5 | Trifluoroalkyldimethyl trimethylsiloxysilicic acid | — | — | — | — | — | — |
| | 6 | (Trimethylsiloxysilicic acid/dimethiconol) cross- | — | — | — | — | — | — |
| | | polymer | | | | | | |
| | 7 | (Dimethicone/vinyldimethicone) cross-polymer | — | — | — | — | — | — |
| | 8 | (Styrene/acrylates) copolymer | — | — | — | — | — | — |
| | 9 | (Cyclohexyl methacrylate/ethylhexyl metacrylate) | — | — | — | — | — | — |
| | | copolymer | | | | | | |
| | 10 | Dextrin isostearate | — | — | — | — | — | — |
| | 11 | PVP | — | — | — | — | — | — |
| (B) | 12 | Ethanol | 10 | 10 | 10 | 10 | 10 | 10 |
| | 13 | Glycerin | — | — | — | — | — | — |
| (C) | 14 | Sodium chloride | 1 | 1 | 1 | 1 | 1 | 1 |
| | 15 | Sodium citrate pKa1 = 3.09 pKa2 = 4.75 pKa3 = 6.41 | — | — | — | — | — | — |
| | 16 | Ascorbyl phosphate Mg | — | — | — | — | — | — |
| (D) | 17 | Dimethyl polysiloxane (25° C., 6 mm²/s) | 3 | — | — | — | — | 7 |
| | 18 | Dimethyl polysiloxane (2° C., 20 mm²/s) | — | 10 | — | — | — | — |
| | 19 | Hydrogenated polyisobutene (25° C., 6 mm²/s) | — | — | 10 | — | — | — |
| | 20 | Isotridecyl isononanate (25° C., 11 mm²/s) | — | — | — | 10 | — | — |
| | 21 | Dimethyl polysiloxane (25° C., 100 mm²/s) | — | — | — | — | 10 | — |
| | 22 | Dimethyl polysiloxane (25° C., 300 mm²/s) | — | — | — | — | — | 3 |
| | 23 | Dimethyl polysiloxane (25° C., 1.5 mm²/s) (Volatile) | — | — | — | — | — | — |
| | 24 | Water | Remainder | Remainder | Remainder | Remainder | Remainder | Remainder |
| | 25 | Phenoxyethanol | 0.3 | 0.3 | 0.3 | 0.3 | 0.3 | 0.3 |
| | | B/A | 3.3 | 3.3 | 3.3 | 3.3 | 3.3 | 3.3 |
| | | C/A | 0.33 | 0.33 | 0.33 | 0.33 | 0.33 | 0.33 |
| | | A/D | 1.00 | 0.30 | 0.30 | 0.30 | 0.30 | 0.43 |
| | | Evaluation | | | | | | |
| | | Film formation speed | ⊚ | ◯ | ⊚ | ◯ | ◯ | ◯ |
| | | Feel of use without stickiness | ⊚ | ◯ | ◯ | ◯ | ◯ | ◯ |
| | | Clogging prevention effect | ◯ | ⊚ | ⊚ | ⊚ | ◯ | ◯ |
| | | Makeup protection | ◯ | ◯ | ◯ | ◯ | ◯ | ◯ |
| | | Fineness of mist | ◯ | ⊚ | ⊚ | ⊚ | ◯ | ◯ |

TABLE 3

| | | | Example | Example |
|---|---|---|---|---|
| | No. | Name of ingredient | 27 | 28 |
| (A) | 1 | Trimethylsiloxysilicic acid | 3 | 3 |
| | 2 | Polymethylsilsesquioxane | — | — |
| | 3 | (Dimethicone/vinyltrimethylsiloxysilicic acid) cross- | — | — |
| | | polymer | | |
| | 4 | (Acrylates/dimethicone) copolymer | — | — |
| | 5 | Trifluoroalkyldimethyl trimethylsiloxysilicic acid | — | — |
| | 6 | (Trimethylsiloxysilicic acid/dimethiconol) cross- | — | — |
| | | polymer | | |
| | 7 | (Dimethicone/vinyldimethicone) cross-polymer | — | — |
| | 8 | (Styrene/acrylates) copolymer | — | — |
| | 9 | (Cyclohexyl methacrylate/ethylhexyl metacrylate) | — | — |
| | | copolymer | | |
| | 10 | Dextrin isostearate | — | — |
| | 11 | PVP | — | — |
| (B) | 12 | Ethanol | 10 | 10 |
| | 13 | Glycerin | — | — |
| (C) | 14 | Sodium chloride | 1 | 1 |
| | 15 | Sodium citrate pKa1 = 3.09 pKa2 = 4.75 pKa3 = 6.41 | — | — |
| | 16 | Ascorbyl phosphate Mg | — | — |
| (D) | 17 | Dimethyl polysiloxane (25° C., 6 mm²/s) | 3 | 0.5 |
| | 18 | Dimethyl polysiloxane (2° C., 20 mm²/s) | — | — |
| | 18 | Hydrogenated polyisobutene (25° C., 6 mm²/s) | — | — |
| | 20 | Isotridecyl isononanate (25° C., 11 mm²/s) | — | — |
| | 21 | Dimethyl polysiloxane (25° C., 100 mm²/s) | — | — |
| | 22 | Dimethyl polysiloxane (25° C., 300 mm²/s) | — | — |
| | 23 | Dimethyl polysiloxane (25° C., 1.5 mm²/s) (Volatile) | 10 | 10 |
| | 24 | Water | Remainder | Remainder |
| | 25 | Phenoxyethanol | 0.3 | 0.3 |
| | | B/A | 3.3 | 3.3 |
| | | C/A | 0.33 | 0.33 |
| | | A/D | 1.00 | 6.00 |
| | | Evaluation | | |
| | | Film formation speed | ⊚ | ⊚ |
| | | Feel of use without stickiness | ⊚ | ⊚ |
| | | Clogging prevention effect | ⊚ | ◯ |
| | | Makeup protection | ⊚ | ⊚ |
| | | Fineness of mist | ⊚ | ⊚ |

| | | | Comparative | Comparative | Comparative | Comparative | Comparative | Reference |
|---|---|---|---|---|---|---|---|---|
| | No. | Name of ingredient | 1 | 2 | 3 | 4 | 5 | 1 |
| (A) | 1 | Trimethylsiloxysilicic acid | — | — | 3 | 3 | 3 | 3 |
| | 2 | Polymethylsilsesquioxane | — | — | — | — | — | — |
| | 3 | (Dimethicone/vinyltrimethylsiloxysilicic acid) cross- | — | — | — | — | — | — |
| | | polymer | | | | | | |
| | 4 | (Acrylates/dimethicone) copolymer | — | — | — | — | — | — |
| | 5 | Trifluoroalkyldimethyl trimethylsiloxysilicic acid | — | — | — | — | — | — |
| | 6 | (Trimethylsiloxysilicic acid/dimethiconol) cross- | — | — | — | — | — | — |
| | | polymer | | | | | | |
| | 7 | (Dimethicone/vinyldimethicone) cross-polymer | — | — | — | — | — | — |
| | 8 | (Styrene/acrylates) copolymer | — | — | — | — | — | — |
| | 9 | (Cyclohexyl methacrylate/ethylhexyl metacrylate) | — | — | — | — | — | — |
| | | copolymer | | | | | | |
| | 10 | Dextrin isostearate | — | — | — | — | — | — |
| | 11 | PVP | — | 3 | — | — | — | — |
| (B) | 12 | Ethanol | 10 | 10 | — | — | 10 | 10 |
| | 13 | Glycerin | — | — | — | 10 | — | — |
| (C) | 14 | Sodium chloride | 1 | 1 | 1 | 1 | — | 1 |
| | 15 | Sodium citrate pKa1 = 3.09 pKa2 = 4.75 pKa3 = 6.41 | — | — | — | — | — | — |
| | 16 | Ascorbyl phosphate Mg | — | — | — | — | — | — |
| (D) | 17 | Dimethyl polysiloxane (25° C., 6 mm²/s) | 10 | 10 | 10 | 10 | 10 | — |
| | 18 | Dimethyl polysiloxane (2° C., 20 mm²/s) | — | — | — | — | — | — |
| | 18 | Hydrogenated polyisobutene (25° C., 6 mm²/s) | — | — | — | — | — | — |
| | 20 | Isotridecyl isononanate (25° C., 11 mm²/s) | — | — | — | — | — | — |
| | 21 | Dimethyl polysiloxane (25° C., 100 mm²/s) | — | — | — | — | — | — |
| | 22 | Dimethyl polysiloxane (25° C., 300 mm²/s) | — | — | — | — | — | — |
| | 23 | Dimethyl polysiloxane (25° C., 1.5 mm²/s) (Volatile) | — | — | — | — | — | 10 |
| | 24 | Water | Remainder | Remainder | Remainder | Remainder | Remainder | Remainder |
| | 25 | Phenoxyethanol | 0.3 | 0.3 | 0.3 | 0.3 | 0.3 | 0.3 |
| | | B/A | — | — | 0 | 0 | 3.3 | 3.3 |
| | | C/A | — | — | 0.33 | 0.33 | 0.00 | 0.33 |
| | | A/D | 0.00 | 0.00 | 0.30 | 0.30 | 0.30 | — |
| | | Evaluation | | | | | | |
| | | Film formation speed | — | Δ | X | X | X | ⊚ |
| | | Feel of use without stickiness | ⊚ | X | Δ | X | X | ⊚ |
| | | Clogging prevention effect | ⊚ | X | ◯ | ◯ | Δ | X |
| | | Makeup protection | X | Δ | Δ | Δ | Δ | ◯ |
| | | Fineness of mist | ⊚ | ⊚ | ⊚ | Δ | Δ | ⊚ |

The compounds employed in Table 1 to 3 are shown below.

### <Lipophilic Resin>

The contents of the lipophilic resins shown in Tables 1 to 3 are the amounts of the solid contents. In such amounts of the solid contents, the commercial products shown below were incorporated in each composition.

A commercial product given as a uniform dispersion by dissolving or swelling in an oil agent was handled as a lipophilic oil agent. One which enabled dissolution by adding to and stirred in 100 mL of an oil agent (temperature of 10 to 20° C.) such as a volatile oil agent and the like at a solid content of 10% concentration was handled as a lipophilic resin. For example one which can be dissolved in any oil agent (for example, dimethicone and the like) employed as a solvent for a commercial product as the ingredient (A) described above can be handled as a lipophilic resin. The following compounds corresponded to lipophilic resins. When the film formation judgment described in <2-1. Lipophilic resin> described above was conducted using a resin liquid obtained by dissolution in an oil agent similar to that described above or a commercial resin liquid, all of the compounds shown below exhibited film formation, and were confirmed to be lipophilic resin having a film-forming ability. The following compounds were able to be used in Formulation Examples 1 to 6 (Examples 29 to 34) as lipophilic resins in Examples 29 and later, and the lipophilic resin contents in Formulation Examples are the amounts of the solid contents.

### [Lipophilic Resin]

1) Trimethylsiloxy silicate: KF-9021, manufactured by Shin-Etsu Chemical Co., Ltd. (solid content 50%, solvent: cyclopentasiloxane)
2) Polymethyl silsesquioxane: SILFORM FLEXIBLE RESIN, manufactured by Momentive Performance Materials Japan LLC. (solid content 100%)
3) (Dimethicone/vinyl dimethicone) cross-polymer: KSG-16, manufactured by Shin-Etsu Chemical Co., Ltd. (solid content 25%, solvent: dimethicone)
   (Acrylates/dimethicone) copolymer: KP545, manufactured by Shin-Etsu Chemical Co., Ltd. (solid content 30%, solvent: cyclopentasiloxane)
4) Trifluoroalkyldimethyl trimethylsiloxy silicate: XS66-B8226, manufactured by Momentive Performance Materials Japan LLC. (solid content 50%, solvent: cyclopentasiloxane)
5) (Trimethylsiloxy silicate/dimethiconol) cross-polymer: DOWSIL FC-5004DM (1.5 cSt) Silicone Resin Gum, manufactured by Dow Corning Toray Co., Ltd. (solid content 40%, solvent: dimethicone)
6) (Dimethicone/vinyl dimethicone) cross-polymer: KSG-16, manufactured by Shin-Etsu Chemical Co., Ltd. (solid content 25%, solvent: dimethicone)
7) (Stylene/acrylates) copolymer: Nissetsu U-3710A (manufactured by Nippon Carbide Industries Co., Inc)
8) (Cyclohexyl methacrylate/ethylhexyl methacrylate) copolymer: PLAS-SIZE L-250, manufactured by Goo Chemical Co., Ltd. (solid content 40%, solvent: light liquid isoparaffin)
9) Dextrin isostearate: UNIFILMA HVY, manufactured by Chiba Flour Milling Co., Ltd. (solid content 100%)

### <Water-Soluble Polymer>

PVP (polyvinylpyrrolidone) LUVISLOL K90 POWDER, manufactured by Nikko Chemicals Co., Ltd.

### <Metal Salt>

Chlorinated Na: sodium chloride, manufactured by Iwaki Co., Ltd.
Citric acid Na (pKa1=3.09 pKa2=4.75 pKa3=6.41): sodium citrate, manufactured by Japan Alcohol Trading Co., Ltd.
Ascorbyl phosphate Mg: C-MATE, manufactured by Wako Pure Chemical Industries, Ltd.

### <Non-Volatile Oil Agent>

The oil agents described in [Low-viscosity non-volatile oil agent] and [High-viscosity non-volatile oil agent] shown below which were employed in Tables 1 to 3 were obtained as non-volatile oil agents which were liquid at 25° C. under normal pressure.

### [Low-Viscosity Non-Volatile Oil Agent]

Dimethyl polysiloxane (25° C., 6 mm²/s): Silicone KF-96 (6cs), manufactured by Shin-Etsu Chemical Co., Ltd.
Dimethyl polysiloxane (25° C., 20 mm²/s): Silicone KF-96 (20cs), manufactured by Shin-Etsu Chemical Co., Ltd.
Hydrogenated polyisobutene (25° C., 6 mm²/s): PARLEAM6, manufactured by NOF CORPORATION.
Isononane tridecyl (25° C., 11 mm²/s): SALACOS913, manufactured by The Nisshin OilliO Group, Ltd.
Dimethyl polysiloxane (25° C., 100 mm²/s): Silicone KF-96 (100cs), manufactured by Shin-Etsu Chemical Co., Ltd.

### [High-Viscosity Non-Volatile Oil Agent]

Dimethyl polysiloxane (25° C., 300 mm²/s): KF-96-300CS

### <Volatile Oil Agent>

Dimethyl polysiloxane (25° C., 1.5 mm²/s): TMF-1.5, manufactured by Shin-Etsu Chemical Co., Ltd.

### (Production Method)

(A) Oil layer: (1) to (10), (17) to (23) are heated (90 to 95° C.) and mixed uniformly.
(B) Water layer: (11) to (16), (24), (25) are mixed uniformly.
(C) Into a push-type container with a spray nozzle (spray aperture ¢ 0.3 mm: non-aerosol container), (B) water layer and (A) oil layer were charged in this order to obtain a composition (a multi-layer makeup protective cosmetic).

It is also possible to charge (1) to (25) in random order into a container with a spray nozzle which is then shaken vigorously to obtain a composition (a multi-layer makeup protective cosmetic).

### <Method for Using Composition>

The composition employed in this test is employed by spraying over the face having a basemake such as foundation and the like or a makeup such as eye shadow, blush and the like. The composition employed in this test is shaken 10 times and then sprayed 10 times all over the face having applied makeup thereby applying onto the face. The composition employed in this test is evaluated by the following evaluation method, and in case for example of the evaluation method of “((b) Feel of use without stickiness” the evaluation is conducted after 5 minutes elapsed (being dried) after spraying.

### (Evaluation Method)

Ten special panelists for cosmetic product evaluation used each of the multi-layer makeup protection products of the test examples, in which the product was sprayed ten times all over the face with makeup (foundation and eye makeup), and “(a) cosmetic film formation speed”, “(b) feel of use without stickiness”, “(c) clogging prevention effect”, “(d) makeup protection effect”, “(e) fineness of the mist” were evaluated and scored on a 5-point scale according to the following evaluation criteria. With respect to the items (a), (b) and (e), evaluation was made during and immediately after use, whereas, with respect to (d), the panelists led normal lives after use, and, after 8 hours, the lasting of makeup was evaluated. With respect to item (c), the spray quantity at the initiation of use and the spray quantity at the termination of use were compared. Subsequently, the average score of all the panelists was calculated, and judgement was made according to the following judgement criteria.

### [Evaluation Criteria]

(a) Cosmetic film formation speed: How fast the cosmetic film is formed after application?

### [Score]: [Evaluation Results]

4: Within 5 minutes after application, cosmetic film was formed.
3: Within 5 to 7 minutes after application, cosmetic film was formed.
2: Within 7 to 9 minutes after application, cosmetic film was formed.
1: Within 9 to 10 minutes after application, cosmetic film was formed.
0: Even at 10 minutes after application, no cosmetic film was formed.

After shaking the container with a spray nozzle charged with the composition ten times, the composition was sprayed 10 times onto a glass plate (5 cm×5 cm), and the film formation of the composition applied onto the glass plate was observed.

(b) Feel of use without stickiness: Any feel of stickiness 5 minutes after application onto skin?

### [Score]: [Evaluation Results]

4: Not sticky at all.
3: Almost not sticky.
2: Barely sticky.
1: Slightly sticky.
0: Extremely sticky.

(c) Clogging prevention effect: During storage at 5° C., 5-push discharge was conducted everyday at 5° C., and the averaged mass of 5-push discharge after the end of the 30-th day based on the averaged mass of 5-push discharge at the start of the test is the ratio shown below (average of N=3).

### [Score]: [Evaluation Results]

4: 95% or more
3: Less than 95% to 80% or more
2: Less than 80% to 65% or more
1: Less than 65% to 50% or more
0: Less than 50%

(c) Clogging prevention effect: Specifically, the composition employed in the test was charged into each of 3 containers with spray nozzles to provide 3 containers with spray nozzles charged with the same composition. They were stored for 10 to 12 hours at 5° C. After storage, each container was measured for the discharged mass (mg) sprayed into a beaker by 5 pushes under the environment of 5° C. The total mass of the 3 containers was calculated, and, from this total mass, the average of the discharged mass (mg) at the test initiation (1st day) per one container was calculated. After initiation of the test, these 3 containers were stored continuously at 5° C., and 5-push discharge was conducted every day under the environment of 5° C. The termination day was the 30-th day, and the discharged mass (mg) sprayed into a beaker by 5-push discharge at the end of the 30-th day was measured, and the average of the discharged mass (mg) at the test termination (30-th day) per one container was calculated similarly to the test initiation (average of N=3). Then, the ratio of the averaged mass of 5-push discharge at the test termination to the averaged mass of 5-push discharge at the test initiation (namely, [the average of the discharged mass at the test termination per one container/the average of the discharged mass at the test initiation per one container]×100(%)) was calculated, and the score at which this ratio corresponds to the evaluation result range (ratio) shown below is the evaluation of the composition employed in the test. A smaller clogging of the spray opening or the spray nozzle attributable to the composition employed leads to a smaller difference between the discharged mass at the test termination and the discharged mass of the test initiation, allowing the evaluation results to approximate 100%.

(d) Makeup protection effect: Any smudging of makeup 8 hours after application?

### [Score]: [Evaluation Results]

4: After 8 hours from application, no smudging is seen at all.
3: After 8 hours from application, almost no smudging is seen.
2: After 8 hours from application, slight smudging is seen.
1: After 8 hours from application, smudging is seen.
0: After 8 hours from application, extreme smudging is seen.

(e) Fineness of the mist: Any feel of the finesse of the mist when applying?

### [Score]: [Evaluation Results]

4: Mist particles are extremely fine.
3: Mist particles are moderately fine.
2: Mist particles are slightly large.
1: Mist particles are large and form water droplets on the skin.
0: Not discharged in mist form.

### [Judgement Criteria]

### [Average Score]: [Judgment]

| | |
|---|---|
| 3.5 or more | ⊚ (Excellent) |
| 2.5 or more to less than 3.5 | ◯ (Good) |
| 1.5 or more and less than 2.5 | Δ (Slightly poor) |
| Less than 1.5 | X (Poor) |

The compositions of Examples 1 to 28 and Reference Example 1 were multilayer makeup protectors having multilayers consisting of upper oil layers and lower water layers.

The compositions of Examples 1 to 28 showed viscosities of 1000 mPa·s or less at 25° C. The viscosity of a composition was measured using a Brookfield viscometer described above in “viscosity of composition of present embodiment (mPa·s)” after shaking the composition 10 times and then allowing to stand.

As evident from the results shown in Tables 1 to 3, the compositions of the present technique (Examples 1 to 28, Reference Example 1) can be sprayed in the form of a fine mist softly all over the face and accordingly they do not spoil the applied makeup, and allow a cosmetic film to be formed all over the face evenly in a suitable thickness even by an ordinary user. Also since the compositions of the present technique caused no smudging over a period as long as 8 hours after application, they have advantageously high sebum resistance and high skin protection effect. These compositions preferably contain volatile oil agents, have a water content, when containing water, within the range of about 50 to 85% by mass, and adjustment within the range of 1 to 95% by mass is considered to be possible.

Aso since the protective compositions of the present technique (Examples 1 to 28) contains the ingredient (A) lipophilic resin (film-forming resin), the ingredient (B) ethanol, the ingredient (C) metal salt and the ingredient (D) low-viscosity non-volatile oil agent, they were excellent in terms of the fineness of the mist when sprayed, the makeup protection effect, the film formation speed, the feel of use without stickiness, the clogging prevention effect. A preferred used mass ratio (B)/(A) is within the range of 1 to 200, more preferably 2 to 100. A preferred used mass ratio (C)/(A) is within the range of 0.1 to 20, more preferably 0.2 to 10. A preferred used mass ratio (A)/(D) is 0.01 to 6, more preferably 0.1 to 3.

While a protective composition containing a hydrophilic resin, a volatile oil agent and water is not preferred in terms of the absence of the stickiness and the absence of the sense of burden (see Patent Literature 4), the protective composition of Reference Example 1 containing the ingredient (C) metal salt exhibited a feel of use without stickiness judged as “⊚(excellent)”. However, the protective composition of Reference Example 1 exhibited no clogging prevention effect. The compositions of Comparatives 1 to 5 also exhibited no makeup protection effect, with other items being evaluated to be poor.

The protective composition of the present technique (for example, Examples 1) exhibited, as a result of exchange of the volatile oil agent into the ingredient (D) low-viscosity non-volatile oil agent when compared to the protective composition of Reference Example 1, a drastically improved clogging prevention effect, which was improved from “x (poor)” to “⊚(excellent)”. Also as a result of a low-viscosity non-volatile oil agent contained in combination with the volatile oil agent, such items as the fineness of the mist when sprayed, the makeup protection effect, the feel of use without stickiness, and the spray opening or spray nozzle clogging prevention effect became excellent without reducing the clogging prevention effect.

With regard to the low viscosity (25° C.: a kinematic viscosity) of the non-volatile oil agent, use of the non-volatile oil agent of 100 mm²/s or less (Examples 25) allowed such items as the fineness of the mist when sprayed, the makeup protection effect, the feel of use without stickiness and the clogging prevention effect to become excellent. Also when using the low viscosity non-volatile oil agent further incorporated with a non-volatile oil agent having a higher viscosity, such items as the fineness of the mist when sprayed, the makeup protection effect, the feel of use without stickiness, and the spray opening or spray nozzle clogging prevention effect became excellent, possibly because the kinematic viscosity of the entire non-volatile oil agents as a combination of multiplicity thereof was substantially lower than that less than 300 mm²/s, or was about 100 mm²/s or less.

Accordingly, the protective composition of the present technique allowed, as a result of containing the ingredient (A) lipophilic resin (film-forming resin), the ingredient (B) ethanol, the ingredient (C) metal salt and the ingredient (D) low-viscosity non-volatile oil agent, such items as the fineness of the mist when sprayed, the makeup protection effect, the feel of use without stickiness, and the spray opening or spray nozzle clogging prevention effect to become excellent. Also as a result of allowing water and/or a volatile oil agent to be further contained, such items as the fineness of the mist when sprayed, the makeup protection effect, the feel of use without stickiness, and the spray opening or spray nozzle clogging prevention effect became extremely excellent.

### Formulation Example

Formulation Example 1: multilayer makeup protection lotion

| (Ingredient) | (Mass %) |
|---|---|
| 1. Dimethyl polysiloxane (*1) | 15 |
| 2. (Dimethicone/vinyl trimethylsiloxy silicate) cross-polymer | 1 |
| 3. Polymethyl silsesquioxane (*2) | 1 |
| 4. Dimethyl polysiloxane (*3) | 2 |
| 5. Methylphenyl polysiloxane (*4) | 1.5 |
| 6. Phytosteryl macadamiate | 0.1 |
| 7. Tocopherol | 0.006 |
| 8. Fragrance | 0.15 |
| 9. Glyceryl tri(2-ethylhexanoate) | 1 |
| 10. Ethanol | 15 |
| 11. Tripropylene glycol (*5) | 3 |
| 12. Dipropylene glycol | 2 |
| 13. Sodium hyaluronate (*6) | 0.1 |
| 14. Collagen (*7) | 0.1 |
| 15. Sodium chloride | 1 |
| 16. Sodium hydrogen phosphate | 0.07 |
| 17. Sodium dihydrogen phosphate | 0.07 |
| 18. Purified water | Remainder |

(*1): KF-96L-5CS (25° C.: 2 mm²/s, manufactured by Shin-Etsu Chemical Co., Ltd.)
(*2): BELSIL PMS MK Powder (manufactured by Wacker Asahikasei Silicone Co., Ltd.)
(*3): KF-96L-10CS (25° C.: 2 mm²/s, manufactured by Shin-Etsu Chemical Co., Ltd.)
(*4): KF-56 (manufactured by Shin-Etsu Chemical Co., Ltd.)
(*5): TPG (manufactured by ADEKA)
(*6): Hyaluronic acid FCH201 (manufactured by Kikkoman Biochemifa Company)
(*7): PANCOGEN MARINE (manufactured by GATTEFOSS)

### (Production Method)

(A) The ingredients (1) to (9) were heated to 95° C. and mixed uniformly.
(B) The ingredients (10) to (18) were mixed uniformly.
(C) The container with a spray nozzle (§ 0.3 mm: non-aerosol container) was charged with Layer (B) and Layer (A) in this order to obtain a multilayer makeup protection lotion.

Formulation Example 1 is a multilayer composition containing the ingredients (A) to (D) and water, and its fineness of the mist when sprayed, makeup protection effect, film formation speed, feel of use without stickiness and clogging prevention were all excellent. When this composition was used, the container containing this composition was shaken for mixing before spraying. This composition, after being allowed to stand, was a multilayer makeup protector having 2 separated layers, namely, an upper oil layer and a lower water layer.

Formulation Example 2: multilayer makeup protection lotion

| (Ingredient) | (Mass %) |
|---|---|
| 1. Isododecane | 3 |
| 2. (Trimethylsiloxy silicate/dimethiconol) cross-polymer (*8) | 3 |
| 3. Tifluoroalkyldimethyl trimethylsiloxy silicate(*9) | 2 |
| 4. Dimethicone (*10) | 2 |
| 5. Dimethicone (*11) | 10 |
| 6. Hydrogenated polydecene (*12) | 2 |
| 7. Phytosterols | 0.05 |
| 8. Fragrance | 0.3 |
| 9. Glyceryl tri(2-ethylhexanoate) | 0.5 |
| 10. BG | 5 |
| 11. Dipropylene glycol | 3 |
| 12. Ethanol | 15 |
| 13. Glycerin | 1 |
| 14. Pyridoxine hydrochloride | 0.1 |
| 15. Sodium chloride | 0.5 |
| 16. Sodium citrate | 0.07 |
| 17. Citric acid | 0.07 |
| 18. Magnesium ascorbyl phosphate | 0.1 |
| 19. Phenoxyethanol | 0.007 |
| 20. Disodium edetate | 0.02 |
| 21. Purified water | Remainder |
| 22. Bentonite (*13) | 0.5 |
| 23. Barium sulfate (*14) | 3 |
| 24. Methyl methacrylate cross-polymer (*15) | 1 |

(*8): DOWSIL FC-5004DM (1.5 cSt) Silicone Resin Gum (manufactured by Dow Corning Toray Co., Ltd., solid content 40%, solvent: dimethicone)
(*9)XS66-B8226 (manufactured by Momentive Performance Materials Japan LLC. (solid content 50%, solvent: cyclopentasiloxane)
(*10): KF-96L-2CS (25° C.: 2 mm²/s, manufactured by Shin-Etsu Chemical Co., Ltd.)
(*11): KF-96L-6CS (25° C.: 2 mm²/s, manufactured by Shin-Etsu Chemical Co., Ltd.)
(*12): SILKFRO 364 (manufactured by LIPO CHEMICALS)
(*13): Kunipia G-4 (manufactured by Kunimine Industries Co., Ltd.)
(*14): Plate-type barium sulfate•H (manufactured by Sakai Chemical Industry Co., Ltd.)
(*15): MX-3000C (manufactured by Soken Chemical & Engineering Co., Ltd.)

### (Production Method)

(A) The ingredients (1) to (9) were mixed uniformly.
(B) The ingredients (10) to (24) were mixed uniformly.
(C) A finger spray container ((0.3 mm: non-aerosol container) was charged with Layer (B) and Layer (A) in this order to obtain a multilayer makeup protection lotion.

Formulation Example 2 is a multilayer composition containing the ingredients (A) to (D) and water, and its fineness of the mist when sprayed, makeup protection effect, film formation speed, feel of use without stickiness and clogging prevention were all excellent. When this composition was used, the container containing this composition was shaken for mixing before spraying. This composition, after being allowed to stand, was a multilayer makeup protector having 3 separated layers, namely, an upper oil layer, a lower water layer and a powder layer on the bottom of the lower layer.

Formulation Example 3: Makeup keeping spray

| (Ingredient) | (Mass %) |
|---|---|
| 1. Trimethylsiloxy silicate (*1) | 1.5 |
| 2. (Acrylates/behenyl acrylate/dimethicone methacrylate) | 0.3 |
| copolymer (*2) | |
| 3. Dimethyl polysiloxane (*3) | 5 |
| 4. Propylene Glycol Dicaprylate | 3 |
| 5. Ethylhexyl methoxycinnamate | 7 |
| 6. Diethylamino hydroxybenzoyl hexyl benzoate | 1.5 |
| 7. Fragrance | 0.3 |
| 8. Polyquaternium-51 (*4) | 2 |
| 9. Bis-PEG-18 methyl ether dimethyl silane (*5) | 1 |
| 10. 1,3-Butylene glycol | 3.5 |
| 11. Purified water | Remainder |
| 12. Ethanol | 20 |
| 13. Sodium chloride | 1 |
| 14. Phenoxyethanol | 0.3 |

Formulation Example 3:
(*1): KF-9021 (50% cyclopentasiloxane solution, manufactured by Shin-Etsu Chemical Co., Ltd.)
(*2): KP-562P (melting point: 45 to 55° C., manufactured by Shin-Etsu Chemical Co., Ltd.)
(*3): KF-96L-10CS (25° C.: 2 mm²/s, manufactured by Shin-Etsu Chemical Co., Ltd.)
(*4): LIPIDURE-PMB (BG) (manufactured by NOF CORPORATION)
(*5): 2501 Cosmetic Wax (melting point 30° C., manufactured by Dow Corning Toray Co., Ltd.)

### (Production Method)

(A) Oil layer: The ingredients (1) to (7) were heated and mixed uniformly.
(B) Water layer: The ingredients (8) to (14) were mixed uniformly.
(C) Layer (A) and Layer (B) were mixed to obtain a stock solution.
(D) 45 Parts of the stock solution of (C) were combined with 55 parts of a propellant (dimethyl ether) and charged in an aerosol container to obtain a makeup keeping spray.

Formulation Example 3 is a multilayer composition containing the ingredients (A) to (D) and water, and its fineness of the mist when sprayed, makeup protection effect, film formation speed, feel of use without stickiness and clogging prevention were all excellent. When this composition was used, the container containing this composition was shaken for mixing before spraying. This composition, after being allowed to stand, was a multilayer makeup protector having 2 separated layers, namely, an upper oil layer and a lower water layer. When this multilayer composition was charged in a non-aerosol container employed in Example 1 and then used, the fineness of the mist when sprayed, the makeup protection effect (skin protection effect), the smooth feed when used, the shine prevention effect and the phase separation performance were all favorable.

Formulation Example 4: Multi-layer makeup
protective cosmetic (Overcoat mist)

| (Ingredient) | (Mass %) |
|---|---|
| 1. Trimethylsiloxy silicate | 4 |
| 2. Ethylhexl methoxycinnamate | 5 |
| 3. Isododecane | 1 |
| 4. Dimethyl polysiloxane(*1) | 10 |
| 5. Dimethyl polysiloxane(*2) | 2 |
| 6. Diphenylsiloxyphenyl trimethicone | 3 |
| 7. Fragrance | 0.15 |
| 8. Sodium chloride | 1 |
| 9. Menthol | 0.5 |
| 10. 1,3-Butylene glycol | 5 |
| 11. Purified water | Remainder |
| 12. Ethanol | 10 |
| 13. Aluminum magnesium silicate (*3) | 0.5 |
| 14. Titanium oxide (mean particle size: 0.035 μm) (*4) | 1 |
| 15. Titanium oxide (mean particle size: 0.25 μm) | 2 |
| 16. Zinc oxide (mean particle size: 0.025 μm) (*5) | 2 |
| 17. Silica (*6) | 1 |

Formulation Example 4:
(*1)KF-96A-5CS (25° C.: 6 mm²/s, manufactured by Shin-Etsu Chemical Co., Ltd.)
(*2)KF-96A-20CS (25° C.: 6 mm²/s, manufactured by Shin-Etsu Chemical Co., Ltd.)
(*3): SUMECTON SA-2 (manufactured by Kunimine Industries Co., Ltd.)
(*4): SMT-500SAS (mean particle size: 0.035 μm, manufactured by TAYCA Corporation)
(*5): MZ-500 (mean particle size: 0.025 μm, manufactured by TAYCA Corporation)
(*6): Silica microbead P-1505 (manufactured by JGC C&C.)

### (Production Method)

(A) Oil layer: The ingredients (1) to (7) were heated and mixed uniformly.
(B) Water layer: The ingredients (8) to (13) were mixed uniformly.
(C) Powder layer: The ingredients (14) to (17) were mixed uniformly.
(D) A container with a spray nozzle ((0.3 mm: non-aerosol container) was charged with (A) oil layer, (B) water layer and (C) powder layer to obtain a multi-layer makeup protective cosmetic (overcoat mist).

Formulation Example 4 is a multilayer composition containing the ingredients (A) to (D) and water, and its fineness of the mist when sprayed, makeup protection effect, film formation speed, feel of use without stickiness and clogging prevention were all excellent. When this composition was used, the container containing this composition was shaken for mixing before spraying. This composition, after being allowed to stand, was a multilayer makeup protector having 3 separated layers, namely, an upper oil layer, a lower water layer and a powder layer on the bottom of the lower layer.

Formulation Example 5: Multilayer makeup protection lotion

| (Ingredient) | (Mass %) |
|---|---|
| 1. Dimethyl polysiloxane(*1) | 15 |
| 2. (Dimethicone/vinyl trimethylsiloxy silicate) cross-polymer | 6 |
| 3. Trimethylsiloxy silicate (*2) | 1.5 |
| 4. Dimethyl polysiloxane (*3) | 5 |
| 5. Jojoba seed oil (*4) | 1.5 |
| 6. (Dimethicone/vinyl dimethicone) cross-polymer (*5) | 0.5 |
| 7. Triethylhexanoin | 1 |
| 8. Fragrance | 0.1 |
| 9. (Acrylates/alkyl (C1-18) acrylate acrylamide) copolymer | 1 |
| 10. Ethanol | 5 |
| 11. Centella Asiatica extract | 1 |
| 12. 1,3-Butylene glycol | 2 |
| 13. Glycerin | 1 |
| 14. Bis-PEG-18 methyl ether dimethylsilane (*6) | 1 |
| 15. Sodium chloride | 1 |
| 16. Sodium monohydrogen phosphate | 0.07 |
| 17. Sodium dihydrogen phosphate | 0.07 |
| 18. Purified water | Remainder |

(*1)BELSIL DM 1 PLUS (25° C.: 1 mm²/s, manufactured by Wacker Asahikasei Silicone Co., Ltd.)
(*2): BELSIL PMS MK Powder (manufactured by Wacker Asahikasei Silicone Co., Ltd.)
(*3): KF-96L-6CS (25° C.: 2 mm²/s, manufactured by Shin-Etsu Chemical Co., Ltd.)
(*4): Organic purified jojoba oil (manufactured by Koei Kogyo Co., Ltd.)
(*5): KSG-016F (solid content 25%: manufactured by Shin-Etsu Chemical Co., Ltd.)
(*6): DOWSIL 2501 COSMETIC WAX (manufactured by Dow Corning Toray Co., Ltd.)

### (Production Method)

(A) The ingredients (1) to (8) were heated to 95° C. and mixed uniformly.
(B) The ingredients (9) to (18) were mixed uniformly.
(C) The container with a spray nozzle (q 0.2 mm: non-aerosol container) was charged with Layer (B) and Layer (A) in this order to obtain a multilayer makeup protection lotion.

Formulation Example 5 is a multilayer composition containing the ingredients (A) to (D) and water, and its fineness of the mist when sprayed, makeup protection effect, film formation speed, feel of use without stickiness and clogging prevention were all excellent. When this composition was used, the container containing this composition was shaken for mixing before spraying. This composition, after being allowed to stand, was a multilayer makeup protector having 2 separated layers, namely, an upper oil layer and a lower water layer.

Formulation Example 6: multiplayer makeup protection lotion

| (Ingredient) | (Mass %) |
|---|---|
| 1. Isododecane | 10 |
| 2. (Cyclohexyl methacrylate/ethylhexyl methacrylate) | 0.5 |
| copolymer | |
| 3. Dextrin isostearate (*2) | 1 |
| 4. Undecane/Tridecane (*3) | 5 |
| 5. Hydrogenated Pentaerythrityl rosinates (*4) | 0.5 |
| 6. Squalane | 0.5 |
| 7. Hydrogenated polyisobutene (*5) | 3.5 |
| 8. Fragrance | 0.06 |
| 9. Hydroxypropyl starch (*6) | 2 |
| 10. Ethanol | 20 |
| 11. Phenoxyethanol | 0.3 |
| 12. 1,3-Butylene glycol | 8 |
| 13. Glycerin | 1 |
| 14. Ethylhexyl Glycerin | 0.05 |
| 15. Sodium chloride | 1 |
| 16. Sodium monohydrogen phosphate | 0.07 |
| 17. Sodium dihydrogen phosphate | 0.07 |
| 18. PVP (*7) | 0.3 |
| 19. Purified water | Remainder |

*1: UNIFILMA HVY (manufactured by Chiba Flour Milling Co., Ltd.)
(*2): Cetiol (Trade Mark) Ultimate (manufactured by BASF Japan Ltd.)
(*3): Ester Gum HP (manufactured by Arakawa Chemical Industries, Ltd.)
(*4): PARLEAM6 (manufactured by NOF CORPORATION)
(*5): Beaute by Roquette (R) ST720 (Manufactured by Roquette Freres)
(*6): PVP K-90 (manufactured by ISP Chemicals)

### (Production Method)

(A) The ingredients (1) to (8) were heated to 95° C. and mixed uniformly.
(B) The ingredients (9) to (19) were mixed uniformly.
(C) The container with a spray nozzle (§ 0.2 mm: non-aerosol container) was charged with Layer (B) and Layer (A) in this order to obtain a multilayer makeup protection lotion.

Formulation Example 6 is a multilayer composition containing the ingredients (A) to (D) and water, and its fineness of the mist when sprayed, makeup protection effect, film formation speed, feel of use without stickiness and clogging prevention were all excellent. When this composition was used, the container containing this composition was shaken for mixing before spraying. This composition, after being allowed to stand, was a multilayer makeup protector having 2 separated layers, namely, an upper oil layer and a lower water layer.

In the present specification, some descriptions are made sometimes with numbers or alphabets such as “1st, 2nd, 3rd . . . ”, “A, B, C . . . ”, “primary, secondary, tertiary . . . ” and the like, which are not intended to allow the present invention to be limited or interpreted narrowly with regard to the order and the like, and the order may be changed arbitrarily. Also in the present specification, “conduct (conducting)” for example of “conduct (conducting) preparation” may be a method, a process, a means or a step, and these terms may be exchanged as appropriate, such as the process being changed to “conduct (conducting)”, the method or the means, and the method being changed to the process, “conduct (conducting)” or the means.

1. A multi-layer makeup protective cosmetic, comprising:
(A) a lipophilic resin,
(B) ethanol,
(C) a metal salt, and
(D) a low-viscosity non-volatile oil agent having a kinematic viscosity of less than 300 mm²/s at 25° C.

2. A method for protecting makeup, which comprises:
applying the makeup onto a surface, and
spraying a multi-layer makeup protective cosmetic onto the surface,
wherein spraying the multi-layer makeup protective cosmetic onto the surface occurs before, during, and/or one or more times after applying the makeup to the surface, and
wherein the multi-layer makeup protective cosmetic comprises:
(A) a lipophilic resin,
(B) ethanol,
(C) a metal salt, and
(D) a low-viscosity non-volatile oil agent having a kinematic viscosity of less than 300 mm²/s at 25° C.

3. The multi-layer makeup protective cosmetic according to claim 1, wherein the lipophilic resin is a silicone-based resin.

4. The multi-layer makeup protective cosmetic according to claim 1, wherein the metal salt is a salt of alkaline metal or a salt of an alkaline earth metal.

5. The multi-layer makeup protective cosmetic according to claim 1, wherein the low-viscosity non-volatile oil agent is one or more kinds selected from the group consisting of a silicone oil, an ester oil and a hydrocarbon oil.

6. The multi-layer makeup protective cosmetic according to claim 1, wherein the mass content ratio (B)/(A) is 1 to 200.

7. The multi-layer makeup protective cosmetic according to claim 1, wherein the mass content ratio (C)/(A) is 0.02 to 20.

8. The multi-layer makeup protective cosmetic according to claim 1, wherein the mass content ratio (A)/(D) is 0.01 to 6.

9. The multi-layer makeup protective cosmetic according to claim 1, wherein the low-viscosity non-volatile oil agent comprises at least a silicone oil.

10. The multi-layer makeup protective cosmetic according to claim 9, wherein the lipophilic resin is a silicone-based resin.

11. The multi-layer makeup protective cosmetic according to claim 1, wherein the low-viscosity non-volatile oil agent comprises at least a dimethyl polysiloxane.

12. The multi-layer makeup protective cosmetic according to claim 1, wherein the kinematic viscosity of the low-viscosity non-volatile oil agent is 3 to 10 mm²/s at 25° C.

13. The multi-layer makeup protective cosmetic according to claim 5, wherein the kinematic viscosity of the low-viscosity non-volatile oil agent is 3 to 10 mm²/s at 25° C.

14. The multi-layer makeup protective cosmetic according to claim 9, wherein the kinematic viscosity of the low-viscosity non-volatile oil agent is 3 to 10 mm²/s at 25° C.

15. The multi-layer makeup protective cosmetic according to claim 1, wherein the mass content ratio (A)/(D) is 0.01 to 0.5.
